(19) United States
(12) Patent Application Publication
(10) Pub. No.: US 2009/0220983 A1
(43) Pub. Date: Sep. 3, 2009

(54) CELL-BASED METHODS FOR DETECTING AND/OR MEASURING BMP-12-RELATED PROTEIN ACTIVITY

(75) Inventors: Joanne Archambault, Arlington, MA (US); Stephen Berasi, Arlington, MA (US); Andrew Dorner, Lexington, MA (US); Scott Jelinsky, Acton, MA (US)

Correspondence Address: WYETH/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 12/393,628

(22) Filed: Feb. 26, 2009

Related U.S. Application Data
(60) Provisional application No. 61/032,525, filed on Feb. 29, 2008.

(51) Int. Cl. C12Q 1/68; C12Q 1/02; G01N 33/567; G01N 33/573

(52) U.S. Cl. 435/6; 435/29; 435/7.21; 435/7.92

(57) ABSTRACT

The invention provides cell-based methods to detect and/or measure the BMP-12-related protein activity of a sample containing a BMP-12-related protein. The methods involve contacting a suitable cell with the sample, and measuring the expression level of at least one BMP-12-related-activity-marker. A dose-dependent increase(s) in the level(s) of the BMP-12-related-activity-markers is indicative of the BMP-12-related protein activity in the sample. The levels of the BMP-12-related-activity-markers of the invention exhibit a dose-responsive increase in response to known BMP-12-related proteins BMP-12, BMP-13, and MP-52, but not to the osteogenic protein, BMP-2.

### PRIOR APPLICATIONS

This application claims the benefit of U.S. Provisional Patent Application No. 61/032,525, filed Feb. 29, 2008, the entire contents of which is hereby incorporated by reference in its entirety.

### DESCRIPTION OF THE INVENTION

1. Background of the Invention

Members of the transforming growth factor-beta (TGF-β) superfamily of proteins possess physiologically important growth-regulatory and morphogenetic properties (Kingsley et al., Genes Dev. 8:133-146 (1994); Hoodless et al., Curr. Topics Microbiol. Immunol. 228:235-272 (1998)). Bone morphogenetic proteins (BMPs) are members of the TGF-β superfamily of growth and differentiation factors (Rosen et al., Principles of Bone Biology 2:919-928 (2002)). Some of the first evidence that BMPs exist was demineralized bone's ability to induce new bone when implanted into muscle (Urist et al., Science 150:893-99 (1965)). BMPs were subsequently biochemically purified from demineralized bone (Wang et al., PNAS 85: 9484-9488 (1988)) and cloned by hybridization of radiolabeled oligonucleotides designed from peptide fragments of the purified proteins (Wozney et al., Science 242:1528-1534 (1988)). Cloned BMPs have been recombinantly expressed and retain their function. For example, recombinant mature BMP-2 (amino acids 283-396) expressed in E. coli exhibits bone stimulating activity both in vitro (Ruppert et al., Eur. J. Biochem. 237:295-302 (1996)) and in vivo (Kübler et al., Int. J. Oral Maxillofacial Surgery 27:305-09 (1998)).

Additional BMPs were cloned by screening for homologues of known BMPs, and have been shown to possess a wide range of activities, including induction of the growth and differentiation of bone, connective, kidney, heart, and neuronal tissues (Rengachary, Neurosug Focus 13(6):1-6 (2002)).

BMP-12-related proteins are a sub-genus of BMPs, which possess tendon and/or ligament-forming activity (Storm et al., Nature 368:639-643 (1994); Wolfman et al., J. Clin. Invest. 100:321-330 (1997); and International Publication No. WO 95/16035). Canonical members include BMP-12, BMP-13, and MP-52, also known as GDFs 7, 6, and 5, respectively. Common sites of tendon or ligament injury include the anterior cruciate ligament (Laurencein et al., Annu. Rev. Biomed. Eng. 1:19-46 (1999)), Achilles' tendon (Mazzone and McCue, Am. Fam. Physician 65:1805-10 (2002)), rotator cuff, and flexor tendon in the hand (Boyer et al., J Hand Ther. 18:80-85 (2005)). Other sources of maladies in tendon or ligament-like tissue include injury, failure, or congenital defects in the ligament-like fascia tissue, which penetrates, supports and surrounds most organs and tissues of the body. Damage to the fascia tissue can result in hernias or organ prolapse, for example bladder, uterus, or rectal prolapse. For discussions of organ prolapse, see Deprest et al., Int. Urogynecol. J. Pelvic Floor Dysfunct. 17:Supp.1:S16-25, and Monga, Curr. Opin. Obstet. Gynecol. 8:366-71 (1996).

In addition to the ability of BMP-12-related proteins to affect ectopic growth of tendon and ligament-like tissue (WO 95/16035; Wolfman et al., 1997; and Helm et al., J. Neurosurg. 95:298-307 (2001)), these proteins have been shown to augment repair of these tissues. For example, BMP-12 improved repair in animal models of rotator cuff (Archambault et al., 5^{th} Comb. Mtg. Ortho. Res. Soc. Canada, USA, Japan, and Europe Podium No:128, (2004)), patellar tendon (Archambault et al., 5^{th} Comb. Mtg. Ortho. Res. Soc. Canada, USA, Japan, and Europe Poster No: 197, (2004)), and flexor profundus tendon (Lou et al., J. Ortho. Res. 19:1199-1202 (2001)). Similarly, MP-52 stimulated healing in an Achilles' tendon defect (Rickert et al., Growth Factors 19:115-26 (2001)). These results suggest that the BMP-12-related proteins may have in vitro and in vivo applications in the treatment of injured tendons and ligaments.

BMPs capable of inducing tendon or ligament-like tissue, including BMP-12, are currently in development as protein-based pharmaceuticals. When protein and DNA are used as pharmaceuticals, a key issue for regulatory agency approval is the ability to produce and standardize batches of the protein or DNA when the batch sizes are increased for large scale manufacturing. One parameter that must be standardized is the biological activity of the pharmaceutical composition. However, routine methods for standardizing large-scale batches of BMP-12 for biological activity have been lacking. For some BMPs, including BMP-2, biological activity may be measured by detecting alkaline phosphatase or osteocalcin induction, or a BMP-response element-luciferase (BRE-luc) reporter construct (for a discussion of BREs, see Kusanagi et al., Mol. Bio. Cell 11:555-65 (2000)). However, the BMP-12-related proteins BMP-12 and BMP-13 are poorly or not at all active in such methods (Wolfman et al., 1997 at page 326; and FIGS. 11 and 13). For example, BMP-12 and BMP-13 are 100-fold less efficient than BMP-2 at inducing the BRE-luc reporter.

Other methods for measuring BMP activity include cell-based assays, in which addition of a BMP changes an observable phenotype of cells, for example, the inhibition of myoblast differentiation of mouse L6 cells (Inada et al., Biochem Biophys. Res. Comm. 222:317-22 (1996)). In ectopic implantation studies, a capsule containing a BMP is implanted into a host animal for 1 to 2 weeks, recovered, and the capsule contents are evaluated histologically for the presence of, for example, bone or tendon-like tissue (U.S. Pat. No. 6,150,328, Example III; Sampath and Reddi, Proc. Natl. Acad. Sci. U.S.A. 80:6591-6595 (1983)). These methods, however, are time-consuming and require subjective analysis of a phenotype, which prevents their use in high throughput or automated screening assays.

Accordingly, a need exists for a rapid, simple, and quantitative method for measuring the activity of BMP-12-related proteins.

The present invention provides simple, rapid, and quantitative cell-based methods for measuring the activity of a BMP-12-related protein in a test sample comprising the protein. The invention is based, in part, on the discovery that thrombospondin-4 (THBS4), a gene expressed principally in tendon, is strongly and rapidly induced in C3H/10T1/2 cells, a mesenchymal cell line, by incubation with a known BMP-12-related protein, BMP-12. Specifically, the levels of THBS4 mRNA in C3H/10T1/2 cells increase in a dose-dependent manner following exposure to BMP-12, BMP-13, or MP-52. In contrast, THBS4 mRNA levels exhibit a “peak” response following treatment with BMP-2, a known osteogenic protein (that is, THBS4 mRNA levels are high in response to low-to moderate BMP-2 concentration, but fall at higher BMP-2 concentrations).

Thus, in one aspect the invention provides methods of detecting and/or measuring BMP-12-related protein activity in a test sample, comprising contacting a cell with the test sample and measuring the expression level of a BMP-12-related-activity-marker. A dose-responsive increase in the level of the BMP-12-related-activity-marker, relative to a cell not contacted with the test sample, indicates the presence of BMP-12-related protein activity in the test sample.

In certain embodiments, the test sample comprises, or is suspected of comprising, a BMP-12-related protein. In certain embodiments the BMP-12-related protein is a recombinant mouse (rm) or human (rh) protein, including rhBMP-12, rmBMP-12, rhBMP-13, rmBMP-13, rhMP-52, and rmMP-52. In particular embodiments, the test sample consists essentially of a BMP-12-related protein, such as rhBMP-12. In some embodiments, the test sample comprises at least one, two, or three proteins selected from the group consisting of BMP-12, BMP-13, and MP-52.

A large variety of cells can be used in the methods provided by the invention. Suitable cell lines include, for example, C3H/10T1/2, 3T3L1, C2C12 murine Clone 14, and primary human mesenchymal stem cells. In particular embodiments, the cell is C3H/10T1/2.

BMP-12-related-activity-markers for use in the invention are markers that show a dose-dependent increase to BMP-12-related proteins, and include THBS4, Tmtc4, AB112350, Birc5, Clu, Ccnb1, AI853363, Fut11, Gm2a, II17d, Matn2, Mia1, Mrps25, Mrps6, Ntrk2, Pbk, Pip, Prlr, Pcp4, Pcx, Rgs7, Ris2, 1500009L16Rik, Sfrp1, 2410017P07Rik, 2810417H13Rik, 3000004C01Rik, 3110001A13Rik, Cytl1, 6230424C14Rik, 9130008F23Rik, 1133, Gas7, D430039N05Rik, Sall1, Sfrp1, Shcbp1, Trps1, and Scaper. In particular embodiments, the BMP-12-related-activity-marker is, for example, THBS4, Pcp4, Prlr, or Pip. In more particular embodiments, the BMP-12-related-activity-marker is THBS4. In some embodiments, the expression level of at least 1, 2, 3, 4, 5, 6, 7, 8, 9, 10, 15, 20, 30, or more different BMP-12-related-activity-markers is measured.

The BMP-12-related-activity-markers provided by the invention include nucleic acids and proteins. In some embodiments, the nucleic acid is a DNA or RNA. In certain embodiments, the RNA is an mRNA. mRNA markers can be detected by means well-known in the art, including Northern blot, cDNA or oligonucleotide microarray, branched DNA assay, PCR, quantitative PCR (q-PCR), and quantitative real-time PCR (qRT-PCR). In particular embodiments the mRNA is detected by qRT-PCR.

Thus, in one embodiment, the invention provides methods of detecting and/or measuring BMP-12-related protein activity in a test sample containing a BMP-12-related protein comprising the steps of contacting a C3H/10T1/2 cell with the test sample and measuring the expression level of THBS4 mRNA by qRT-PCR. A dose-dependent increase in THBS4 mRNA expression levels in the C3H/10T1/2 cell, relative to an untreated control, indicates the presence of BMP-12-related protein activity in the sample.

Suitable BMP-12-related-activity-markers include proteins, or fragments thereof. The protein or fragment may be detected by means well-known in the art, including: Western blot, immunochemistry, ELISA, chromatography, mass spectrometry, and tandem mass spectrometry.

In some embodiments, the methods of the invention further comprise the step of measuring the expression level of at least one osteogenic-marker, where a dose-responsive increase in the ratio of the BMP-12-related-activity-marker expression level to the osteogenic-marker expression level in the cell, compared to the ratio in a cell not contacted with the test sample, indicates the presence of BMP-12-related protein activity in the test sample. In particular embodiments the osteogenic-marker is osteocalcin (OST). In some embodiments, the BMP-12-related-activity-marker is THBS4. In certain embodiments, the ratio of expression levels of the BMP-12-related-activity-marker and osteogenic-marker is probative of the specificity of a BMP-12-related protein activity in the test sample.

In another aspect, the invention provides kits for detecting and/or measuring BMP-12-related protein activity of a test sample. The kit may comprise primers for measuring the expression levels of one or more BMP-12-related-activity-markers and instructions for detecting mRNA levels of the markers. In some embodiments, the kit may comprise antibodies for measuring the expression levels of one or more BMP-12-related-activity-markers and instructions for detecting protein levels of the markers. In certain embodiments the kit may comprise a BMP-12-responsive cells.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a bar graph of THBS4 mRNA levels across several human tissues.

FIG. 2 is a graph which plots cycle threshold (Ct) in a series of qRT-PCR reactions as a function of decreasing THBS4 mRNA for 6 sets of TaqMan™ (qRT-PCR) oligonucleotide probes to mouse THBS4. In vitro-transcribed THBS4 mRNA was included in each reaction in the amount indicated. The average Ct of 3 replicates is plotted.

FIG. 3 is a bar graph which shows the fold-induction of THBS4 mRNA in C3H/10T1/2 cells following incubation for 1, 2, or 3 days at the indicated dose of rhBMP-12, compared to a vehicle control. The THBS4 mRNA was measured by TaqMan™ qRT-PCR using the exon 1-2 probe. The plotted values are the average and standard deviation of three biological replicates per data point.

FIG. 4 is a bar graph which shows the fold-induction of THBS4 mRNA in C3H/10T1/2 cells treated with 100 nM rhBMP-12 for 3 days in the indicated well format, compared to a vehicle control. The plotted values are the average and standard deviation of three biological replicates per data point.

FIG. 5 is a bar graph of the relative fold-induction of THBS4 mRNA in C3H/10T1/2 cells at the indicated density treated with 100 nM rhBMP-12 for 3 days, compared to a vehicle control. The plotted values are the average and standard deviation of three biological replicates per data point.

FIGS. 6A-6D are bar graphs that show the relative fold-induction of THBS4 mRNA in C3H/10T1/2 cells at passage 3 (FIG. 6A), passage 4 (FIG. 6B), passage 5 (FIG. 6C), or passage 16 (FIG. 6D), seeded at 2×10⁵ cells per well of a 12 well plate, and incubated with the indicated dose of rhBMP-12 (nM) for 3 days, compared to a vehicle control. The plotted values are the average and standard deviation of three biological replicates per data point.

FIGS. 7A-7D show the cellular response to untreated BMP-12 or BMP-12 oxidized by treatment with peracetic acid (PAA). FIG. 7A is a fitted semi-logarithmic plot of the relative fluorescent units (RFUs) from a cell-based BMP-responsive element luciferase (BRE-luc) reporter as a function of untreated rhBMP-12 or 0.9 mM PAA-treated BMP-12 concentration. FIG. 7B is a fitted semi-logarithmic plot of the RFUs from a reporter as a function of protein concentration for untreated rhBMP-12 or BMP-12 treated with 1.2 mM PAA. FIG. 7C is a bar graph of the fold-induction of THBS4 mRNA in C3H/10T1/2 cells following a three day treatment with the indicated dose of either PAA-treated or untreated rhBMP-12. The plotted values are the average and standard deviation of three biological replicates per data point. FIG. 7D is a fitted semi-logarithmic plot of the fold-induction of THBS4 mRNA in C3H/10T1/2 cells following a three day treatment with the indicated dose of untreated rhBMP-12 or BMP-12 treated with 0.9 mM or 1.2 mM PAA. The plotted values are the average and standard deviation of three biological replicates per data point.

FIGS. 8A-8D are bar graphs of the fold-induction of THBS4 mRNA in C3H/10T1/2 cells at passage 2 (FIG. 8A), passage 3 (FIG. 8B), passage 4 (FIG. 8C), or passage 5 (FIG. 8D) following a three day treatment with the indicated dose of rhBMP-12. Cells were seeded at 2×10⁵ cells per well of a 12 well plate. The plotted values are the average and standard deviation of three biological replicates per data point. The fold-change of untreated cells was set to 1.

FIGS. 9A-9H are bar graphs of the fold-induction of THBS4 mRNA in C3H/10T1/2 cells at passage 3 (FIGS. 9A, 9B), 4 (FIGS. 9C, 9D), 5 (FIGS. 9E, 9F) or 16 (FIGS. 9G, 9H) following a three day treatment with the indicated dose of rhBMP-12 (FIGS. 9A, 9C, 9E, 9G) or BMP-2 (FIGS. 9B, 9D, 9F, 9H). Cells were seeded at 2×10⁵ cells per well of a 12 well plate. The plotted values are the average and standard deviation of three biological replicates per data point. The fold-change of untreated cells was set to 1.

FIGS. 10A-10B are plots of THBS4 mRNA fold induction of passage 3 C3H/10T1/2 cells treated with BMP-12 (FIG. 10A) or BMP-2 (FIG. 10B) for three days, with overlaid curve fits. The plotted values are the average and standard deviation of three biological replicates per data point. The fold-change of untreated cells was set to 1.

FIGS. 11A-11H are bar graphs of the fold-induction of THBS4 mRNA in C3H/10T1/2 cells at passage 2 (FIGS. 11A, 11B), passage 3 (FIGS. 11C, 11D), passage 4 (FIGS. 11E, 11F), or passage 5 (FIGS. 11G, 11H), following a three day treatment with the indicated dose of rhBMP-12 (FIGS. 11A, 11C, 11E, and 11G) or BMP-2 (FIGS. 11B, 11D, 11F, and 11H). Cells were seeded at 2×10⁵ cells per well of a 12 well plate. The plotted values are the average and standard deviation of three biological replicates per data point. The fold-change of untreated cells was set to 1.

FIGS. 12A-12C show the data from FIGS. 11C, 11E, and 11G, (BMP-12-treated cells) at passages 3 (FIG. 12A), passage 4 (FIG. 12B), and passage 5 (FIG. 12C), plotted with a sigmoidal curve fit.

FIGS. 13A-13H are bar graphs of the fold-induction of osteocalcin (OST) mRNA in C3H/10T1/2 cells at passage 2 (FIGS. 13A, 13B), passage 3 (FIGS. 13C, 13D), passage 4 (FIGS. 13E, 13F), or passage 5 (FIGS. 13G, 13H), following a three day treatment with the indicated dose of rhBMP-12 (FIGS. 13A, 13C, 13E, and 13G) or BMP-2 (FIGS. 13B, 13D, 13F, and 13H). Cells were seeded at 2×10⁵ cells per well of a 12 well plate. The plotted values are the average and standard deviation of three biological replicates per data point. The fold-change of untreated cells was set to 1.

FIGS. 14A-14F are plots of the log₂ ratio of THBS4 to OST mRNA in C3H/10T1/2 cells at passage 3 (FIGS. 14A, 14B), passage 4 (FIGS. 14C, 14D), or passage 5 (FIGS. 14E, 14F), following a three day treatment with the indicated dose of rhBMP-12 (FIGS. 14A, 14C, 14E) or rhBMP-2 (FIGS. 14B, 14D, 14F). Cells were seeded at 2×10⁵ cells per well of a 12 well plate.

FIGS. 15A-15F are plots of the relative fold-induction of THBS4 (FIGS. 15A, 15B) and OST (FIGS. 15C, 15D) mRNA in passage 9 C3H/10T1/2 cells treated for three days with the indicated dose (nM) of rhBMP-12 (FIGS. 15A, 15C, and 15E) or rhBMP-2 (FIGS. 15B, 15D, and 15F). Cells were seeded at 2×10⁵ cells per well of a 12 well plate. The plotted values are the average and standard deviation of three biological replicates per data point. The fold-change of untreated cells was set to 1. The log₂ transformed ratio of THBS4/OST (FIGS. 15E, 15F) induction is also provided.

FIGS. 16A-16B are bar graphs of the fold-induction of THBS4 mRNA in C3H/10T1/2 cells following a three day treatment with the indicated dose of rhBMP-12 (FIG. 16A) or BMP-2 (FIG. 16B). Cells were frozen at passage 7, then recovered and assayed at passage 10, seeded at a density of 2×10⁵ cells per well of a 12 well plate. The plotted values are the average and standard deviation of three biological replicates per data point.

FIG. 17 is a bar graph of the fold-induction of THBS4 mRNA in C3H/10T1/2 cells following a three day treatment with the indicated doses of rhBMP-12 or BMP13. Cells were seeded at a density of 2×10⁵ cells per well of a 12 well plate. The plotted values are the average and standard deviation of three biological replicates per data point.

FIG. 18 is a Western blot for THBS4 protein in culture supernatants. C3H/10T1/2 cells were seeded at 2×10⁵ cells per well of a 12 well plate, incubated with 1, 10, or 100 nM of BMP-2, BMP-12, or BMP-13 and analyzed after 1, 3, or 4 days.

FIGS. 19A-19B are bar graphs of THBS4 (FIG. 19A) and OST (FIG. 19B) mRNA levels, as measured in a branched DNA assay. Passage 6 cells were seeded at 2×10⁵ cells per well of a 12 well plate and incubated with the indicated dose of either BMP-12 or BMP-2, and assayed at day 3. The plotted values are the average and standard deviation of three biological replicates per data point.

FIG. 20 is a bar graph of the fold induction in THBS4 mRNA level in cells treated with BMP-12 for different periods of time. The “starved” treatment group was serum starved for six hours before treatment. Cells were seeded at a density of 2×10⁵ cells per well of a 12 well plate. The plotted values are the average and standard deviation of three biological replicates per data point.

### EXEMPLARY EMBODIMENTS

The present invention provides cell-based methods for detecting and/or measuring BMP-12-related protein activity of a test sample comprising a BMP-12-related protein. The term “BMP-12-related protein activity” refers to one or more known biological activities of BMP-12, and particularly to the tendon or ligament-like tissue inducing activity of BMP-12, such as affecting the morphological changes associated with tendon or ligament-like tissue in a suitable host cell and/or the ability to induce a transcriptional profile characteristic of BMP-12.

In general, the methods of the invention comprise two steps:

1) contacting a cell with a test sample, and

2) measuring the expression level of a BMP-12-related-activity-marker.

A dose-dependent increase in the level of the BMP-12-related-activity-marker in the cell, relative to an untreated cell, indicates the presence of BMP-12-related protein activity in the test sample. To assess whether a BMP-12-related-activity-marker exhibits a dose-dependent increase, typically the test sample is applied to the cells at several concentrations, incubated, and the fold-increase of the BMP-12-related-activity-marker, relative to control cells not treated with the test sample, is determined. That is, a dose-response curve is calculated. The BMP-12-related-activity-markers used in the methods of the invention show a dose-dependent increase to BMP-12-related proteins, such as BMP-12, whereas the osteogenic protein, BMP-2, elicits a dose-dependent peak response.

The shape of a dose response curve is probative of BMP-12-related protein activity—a dose dependent increase corresponds to detecting BMP-12-related protein activity. The position of the dose response curve on the x-axis corresponds to measuring the BMP-12-related protein activity. That is, changes in the concentration of the test sample results in an x-axis translation of a dose-response curve. For example, if a test sample with BMP-12-related protein activity and a 100-fold dilution of the same test sample are measured by the methods of the invention, the dose-response curve of the undiluted sample will be shifted two log-decades to the left (that is, toward a lower concentration) of the diluted sample. This is because less of the undiluted sample is needed to elicit the same increase in levels of the BMP-12-related-activity-marker as the diluted sample.

The fold-increase of a BMP-12-related-activity-marker, such as, for example, THBS4 mRNA, may be calculated by means well-known in the art, including, for example, the ΔΔCt method. Typically the expression level of a gene is calculated relative to some normalizing factor, for example, total RNA, or a “house-keeping” gene, whose level is relatively constant under the testing conditions. In certain embodiments, GUSB (glucuronidase, beta; GeneID: 110006 [Mus musculus], 2990 [Homo Sapiens]) is a useful normalizing gene for THBS4 mRNA, as its mRNA remains at an approximately constant level following treatment with BMP-12. In addition to normalizing levels of a BMP-12-related-activity-marker to, for example, a different transcript from the same cell; fold-induction is calculated relative to a standard, such as cells not treated with the test sample.

### BMP-12 Related Protein Activity

The methods of the present invention are useful for detecting and/or measuring the BMP-12-related protein activity of a test sample. In some embodiments, the sample contains, or is suspected of containing, a BMP or other compound with suspected tendon or ligament-like tissue inducing activity. In other embodiments, the test sample comprises a known BMP-12-related protein whose activity must be confirmed. “BMP-12-related protein” refers to a protein, or fragment thereof, that has at least about 70% amino acid identity to the mature (bioactive) region of a BMP-12, BMP-13, or MP-52 (also known as GDFs 7, 6, and 5) protein, and that possesses tendon or ligament-like tissue inducing activity. In some embodiments, the amino acid sequence of the BMP-12-related protein is at least 75%, 80%, 85%, 90%, 95%, 98%, or 99% identical at the amino acid level to the sequence of the mature region of a BMP-12, BMP-13, or MP-52. BMP-12-related proteins have been identified in numerous species, including mammals such as human, macaque, mouse, and rat. See, for further examples, Table 1, which lists the National Center for Biotechnology Information (NCBI) Entrez GeneID number for BMP-12, BMP-13, and MP-52 from a variety of species. These GeneIDs may be used to retrieve the publicly-available annotated mRNA or protein sequences, for example, at the NCBI world-wide web portal, which may be found at the following uniform resource locator (URL): http://www.ncbi.nim.nih.gov/sites/entrez?db=gene. As an illustration, GeneIDs for human BMP-12, BMP-13, and MP-52 can be used to retrieve the following protein reference sequences: NP_{—}878248.2, NP_{—}001001557.1, and NP_{—}000548.1. Similarly for mouse, the BMP-12, BMP-13, and MP-52 protein reference sequences that can be retrieved include NP_{—}038555.1, NP_{—}038554.1, and NP_{—}032135.1. All information associated with GeneIDs referenced in this application, including, for example, genomic loci, genomic sequences, functional annotations, allelic variants, and reference mRNA and protein sequences are hereby incorporated by reference in their entirety. All reference sequences are also incorporated by referenced in their entirety, including annotated features of the sequences, such as exon boundaries of mRNAs or structural features of proteins.

TABLE 1

| | BMP-12 | BMP-13 | MP-52 |
|---|---|---|---|
| Species | Gene ID | Gene ID | Gene ID |
| Homo sapiens | 151449 | 392255 | 8200 |
| Bos taurus | 286859 | 407175 | 539559 |
| Canis lupus familiaris | 482989 | | 485850 |
| Danio rerio | 30642 | | 30195 |
| Equus caballus | | 100059475 | 100034228 |
| Gallus gallus | | 428655 | 374249 |
| Macaca mulatta | 702462 | 701126 | 705432 |
| Mus musculus | 238057 | 242316 | 14563 |
| Pan troglodytes | 470322 | | 458202 |
| Papio aubis | | | 100137480 |
| Rattus norvegicus | 252833 | 252834 | 252835 |
| Xenopus laevis | | 399144 | 446295 |
| Xenopus tropicalis | | 548831 | 100189547 |

In certain embodiments, the test sample comprises any BMP or growth factor with BMP-12-related protein activity. In one embodiment, the invention provides methods for detecting and/or measuring the BMP-12-related protein activity of a BMP by incubating cells with the BMP or by expressing the BMP transgenically. In particular embodiments, the test sample comprises at least one protein selected from the group consisting of BMP-12, BMP-13, and MP-52. In more particular embodiments, the test sample comprises at least two proteins selected from the group consisting of BMP-12, BMP-13, and MP-52. In yet more particular embodiments, the test sample comprises BMP-12, BMP-13, and MP-52.

BMPs are a highly homologous family of proteins, and are separated into subgroups based on even higher levels of homology. Some important subgroups include: BMP-2 and BMP-4; BMP-5, BMP-6, and BMP-7; and BMP-12, BMP-13, and MP-52. In particular, BMPs share an identifying pattern of cysteine residues in the carboxy-terminal region of the protein, which are needed for BMP activity. Accordingly, the methods of the invention may be used to evaluate the BMP-12-related protein activity of compositions comprising one or more of the following BMPs: BMP-2, BMP-3, BMP-4, BMP-5, BMP-6, and BMP-7 (disclosed, for example, in U.S. Pat. Nos. 5,108,922; 5,013,649; 5,116,738; 5,106,748; 5,187,076; and 5,141,905), BMP-8 (disclosed in PCT WO 91/18098), BMP-9 (disclosed in PCT WO 93/00432), BMP-10 (disclosed in PCT WO 94/26893) BMP-11 (disclosed in PCT WO 94/26892), BMP-12 and BMP-13 (disclosed in PCTWO 95/16035), BMP-15 (disclosed in U.S. Pat. No. 5,635,372), BMP-16 (disclosed in U.S. Pat. No. 6,331,612), MP52 (disclosed in PCT WO 93/16099), and BMP-17 and BMP-18 (disclosed in U.S. Pat. No. 6,027,917), including combinations and heterodimers thereof. A reference to these proteins, should be understood to include variants, allelic variants, fragments of, and mutant BMPs, including but not limited to deletion mutants, insertion mutants, and substitution mutants. In particular, reference to any particular BMP should be understood to include N-terminal truncation fragments where about 1, 3, 5, 7, 10, 15, 20, 25, 30, 35, or more residues have been removed from the N terminus of the mature protein.

In order to establish the dose-response curve for the test sample protein (e.g., a BMP-12-related protein), a range of concentrations of the protein are used in the methods of the invention. In some embodiments, the BMP in the test sample is present at a concentration of about 0.1 nM to about 100 nM. In particular embodiments, the concentrations of a BMP-12-related protein in the test sample is at least about 0.001 nM, 0.005 nM, 0.01 nM, 0.05 nM, 0.1 nM, 0.5 nM, 1 nM, 5 nM, 10 nM, 25 nM, 50 nM, 100 nM, 250 nM, 500 nM, 1000 nM, or more. In some embodiments, a BMP-12-related protein-containing test sample of unknown concentration or activity is used in the methods provided by the invention and compared to a standard curve of, for example, a dilution series of a known concentration of BMP-12-related protein, such as mature rhBMP-12, rhBMP-13, or rhMP-52, in order to determine the concentration and activity of the test sample. In certain embodiments, the dilution series tests at least 3, 4, 5, 6, 7, 8, 9, 10, 12, or more dilutions of the test sample. In particular embodiments, the dilution series tests at least 4 dilutions of the test sample. In more particular embodiments, the dilution series includes test sample protein concentrations of about 0.1 nM, 1.0 nM, 10 nM, and 100 nM.

The test sample protein may be added to a test cell as crude, purified, or recombinant protein. Alternatively, the test sample protein may be expressed by transiently or stably transfecting the test cell with DNA encoding the protein of interest. Finally, the test sample protein may be endogenously expressed by the test cell. Methods for producing crude, recombinant, or purified versions of a BMP-12-related or other protein to be assayed are well known in the art and systems and reagents for producing these proteins for use in the methods of the invention are well known and commercially available from a number of sources. When transfecting cells with DNA encoding a BMP-12-related or other protein, conventional gene transfer methods may be used to introduce DNA into cells.

It is contemplated that any test sample may be tested by the methods provided by the invention. A test sample may include, for example, a peptide, polypeptide, protein, antibody, nucleic acid, carbohydrate, lipid, hormone, vitamin moiety, a low-molecular weight chemical, for example, a pharmaceutical; or a combination of one or more such compounds. Combinations may include conjugated, or unconjugated combinations and mixtures. Test samples may be naturally-occurring (for example, biochemically purified) or synthetic (for example, chemically synthesized or recombinantly produced) compounds. Additionally, the test sample may contain no, some, or all non-native components (for example, non-natural amino acids, blocking or protecting groups).

Test samples may be naturally occurring, chemically modified, or completely synthetic. A test sample may be a simple composition, that is, a single active chemical entity, or a complex mixture, for example, a mixture of any of the test compounds discussed supra. For example, in some embodiments a test sample may include a single compound with known or unknown BMP-12-related protein activity. In another embodiment, two compounds known to have BMP-12-related protein activity could be part of the test sample to investigate possible synergistic effects. In still another embodiment a compound known to have BMP-12-related protein activity is combined with a second test compound to test the second test compound's ability to inhibit or augment activity of the compound known to have BMP-12-related protein activity.

### BMP-12-Related-Activity-Markers

As used herein, a BMP-12-related-activity-marker refers to a nucleic acid or protein whose expression increases in a dose-dependent manner in response to known BMP-12-related proteins BMP-12, BMP-13, and MP-52, but does not increase in a dose-dependent manner in response to the osteogenic protein BMP-2. In this application, expression may refer to either transcription or translation.

The BMP-12-related-activity-markers that may be used in the methods of the invention include: THBS4, Tmtc4, AB112350, Birc5, Clu, Ccnb1, A1853363, Fut11, Gm2a, II17d, Matn2, Mia1, Mrps25, Mrps6, Ntrk2, Pbk, Pip, Prlr, Pcp4, Pcx, Rgs7, Ris2 (also known as Cdt1), 1500009L16Rik, Sfrp1, 2410017P07Rik, 2810417H13Rik, 3000004C01Rik, 3110001A13Rik, Cytl1, 6230424C14Rik, 9130008F23Rik, II33, Gas7, D430039N05Rik (also known as Fam171b), Sall1, Sfrp1, Shcbp1, Trps1, and Scaper. In some embodiments, the methods of the invention comprise the step of measuring the expression level of at least 1, 2, 3, 4, 5, 10, or more, BMP-12-related-activity-markers. Other suitable markers may be identified by the similarity of their expression profiles to known BMP-12-related-activity-markers, such as THBS4, that is, exhibiting a dose-dependent increase in response to BMP-12 and a peak response to BMP-2.

Table 2 contains the Gene symbol, and murine NCBI GeneID for the BMP-12-related-activity-markers that may be used in the methods of the invention. A GeneID can be used to retrieve, for example, curated DNA, mRNA, and protein sequences as well as identify homologs from other organisms for the particular gene. The Entrez gene database can also be queried with the gene symbols listed in Table 2 to identify homologous sequences from other organisms. These GeneIDs and Reference Sequences are incorporated by reference in their entirety.

TABLE 2

| Symbol | Gene ID | mRNA | Symbol | Gene ID | mRNA |
|---|---|---|---|---|---|
| THBS4 | 21828 | NM_011582.2 | Rgs7 | 24012 | NM_011880.3 |
| Tmtc4 | 70551 | NM_028651.2 | Ris2 | 67177 | NM_026014.3 |
| AB112350 | 242864 | NM_178728.5 | 1500009L16Rik | 69784 | NC_000076.5 |
| Birc5 | 11799 | NM_009689.2 | Sfrp1 | 20377 | NM_013834.2 |
| Clu | 12759 | NM_013492.2 | 2410017P07Rik | 103268 | NM_026643.4 |
| Ccnb1 | 268697 | NM_172301.3 | 2810417H13Rik | 68026 | NM_026515.2 |
| AI853363 | 98471 | AI_853363.1 | 3000004C01Rik | 70218 | NM_197959.1 |
| Fut11 | 73068 | NM_028428.2 | 3110001A13Rik | 66540 | NM_025626.4 |
| Gm2a | 14667 | NM_010299.2 | Cytl1 | 231162 | NM_001081106.1 |
| Il17d | 239114 | NM_145837.3 | 6230424C14Rik | 67786 | AK078961 |
| Matn2 | 17181 | NM_016762.2 | 9130008F23Rik | 71583 | NR_024624.1 |
| Mia1 | 12587 | NM_019394.3 | Il33 | 77125 | NM_133775.1 |
| Mrps25 | 64658 | NM_025578.4 | Gas7 | 14457 | NM_001109657.2 |
| Mrps6 | 121022 | NM_080456.1 | D430039N05Rik | 241520 | NM_175514.2 |
| Ntrk2 | 18212 | NM_001025074.1 | Sall1 | 58198 | NM_021390.3 |
| Pbk | 52033 | NM_023209.2 | Sfrp1 | 20377 | NM_013834.2 |
| Pip | 18716 | NM_008843.3 | Shcbp1 | 20419 | NM_011369.2 |
| Prlr | 19116 | NM_011169.4 | Trps1 | 83925 | NM_032000.2 |
| Pcp4 | 18546 | NM_008791.2 | Scaper | 244891 | NM_001081341.1 |
| Pcx | 18563 | NM_008797.2 | | | |

In one embodiment, the BMP-12-related-activity-marker is THBS4. As described in detail below, THBS4 is specifically expressed in tendon, and its mRNA and protein are both highly induced by known BMP-12-related proteins BMP-12, BMP-13, and MP-52. Table 3 lists the NCBI Entrez GeneID number with curated mRNA and protein accession numbers for thrombospondin-4 from a variety of species. These GeneIDs and Reference Sequences are incorporated by reference in their entirety. For reference, SEQ ID NOs:20 and 22 correspond to the human and mouse THBS4 nucleic acid reference sequences, respectively.

TABLE 3

| Species | GeneID | mRNA | Protein |
|---|---|---|---|
| Homo sapiens | 7060 | NM_003248 | NP_303239 |
| Bos taurus | 541281 | NM_001034728 | NP_001029900 |
| Canis lupus | 488930 | XM_546047 | XP_546047 |
| familiaris | | | |
| Danio rerio | 252850 | NM_173226 | NP_775333 |
| Gallus gallus | 396306 | XM_424763 | XP_424763 |
| Macaca mulatta | 574276 | XM_001109898 | XP_001109898 |
| Mus musculus | 21828 | NM_011582 | NP_035712 |
| Pan troglodytes | 450172 | XM_517680 | XP_517680 |
| Rattus norvegicus | 29220 | XM_342172 | XP_342173 |
| Xenopus laevis | 446985 | NM_001093627.1 | NP_001087096.1 |
| Xenopus tropicalis | 780130 | NM_001079205.1 | NP_001072673.1 |

THBS4 assembles as a pentamer and is abundant in tendon (Hauser et al., FEBS letters 368:307-10 (1995)). In equine tendon, THBS4 pentamers include heterooligomers of THBS4 and cartilage oligomeric matrix protein (COMP), which are connected by disulfide bridges (Sodersten and Ekman Con. Tiss. Res. 47:85-91 (2006)). Structural characterization of THBS4 suggests that the pentamers form via their heptad repeats in a coiled-coil configuration (Narouz-Ott et al., J. Biol. Chem. (2000)). The N-terminal domain of THBS4 contains a heparin binding domain, while the C-terminus is necessary for binding both collagenous and non-collagenous proteins. Id. The pentameric structure is not required for THBS4 to bind to other proteins. Id. For additional biophysical characterization of THBS4, see, for example, Misenheimer and Mosher J. Biol. Chem. 280:41229-35 (2006).

### Cells

Cell lines suitable for use in the methods of the invention are well known in the art and widely available. A number of suitable cell lines can be obtained from depositories such as the America Type Culture Collection (ATCC), Manassas, Va.

Cells suitable for use in the invention may be naturally, or engineered to be, BMP-12-responsive, i.e., the cells exhibit a dose-responsive increase in the expression level of at least one BMP-12-related-activity-marker in response to a BMP-12-related protein.

In certain embodiments, suitable cells for performing the inventive method include mesenchymal cells, such as mesenchymal stem cells, and preosteoblastic cells. As is known in the art, undifferentiated mesenchymal cells are able to differentiate along, for example, osteoblastic, chondrocyte, adipocyte, tenocyte, or myocyte pathways to form osteoblasts, chondrocytes, adipocytes, tenocytes, or myocytes. In general, mesenchymal cells suitable for use in the methods of the invention can be any cell line that is capable of differentiating along an osteoblast, tenocyte, or chondrocyte lineage under appropriate conditions, for example, when exposed to the appropriate growth factor(s) or serum.

Preferably, relatively undifferentiated mesenchymal cells are used in the methods of the invention. The cells may be non-expanded primary cells, culture-expanded primary cells, or established cell lines (preferably clonal cell lines). Primary cells are non-immortalized cell lines that are recovered directly from an animal and optionally grown for a limited number of passages. The cells may be derived from adult, infant, fetal, or embryonic sources. The cells may be from a nematode, worm, insect, amphibian, or mammal, for example, human, primate, ovine, bovine, porcine, equine, feline, canine, or rodent source. In particular embodiments, the cells may be human or rodent. In certain embodiments of the invention, the cells are selected from W20-17, C2C12, C3H/10T1/2, MC3T3-E1, Clone 14, 3T3L1, RCJ, 2T3, and ST2 cells. In particular embodiments, the cells are C3H/10T1/2 cells. Numerous primary human mesenchymal cells may also be used in the methods provided by the invention.

Cells useful in the methods of the invention may express the proteins needed to respond to BMP-12-related proteins as part of their native proteomes or recombinantly express one or more of these components. Recombinant molecules can be introduced by routine methods known in the art, and may be stably or transiently expressed, that is, integrated into the genome or maintained on an exogenous plasmid. See, for example, Joseph Sambrook and David Russell, Molecular Cloning: A Laboratory Manual Cold Spring Harbor Laboratory Press; 3rd edition (2001). Thus, cells not otherwise capable of responding to BMP-12-related proteins could be made suitable for use in the methods of the invention by introducing a missing or deficient protein, for example, encoded on a plasmid.

It is theorized, but not relied upon, that the ability of a cell to respond to BMP-12-related proteins, for example, BMP-12, BMP-13, or MP-52, requires expression of a Type I receptor (for example, one of the activin receptor-like kinases: ALK3 or ALK6), a Type II receptor (for example, BMPRII or ActRIIA), a receptor-associated Smad (for example, any one of Smads 1, 5, or 8), and the co-Smad: Smad4. Hoffmann et al. (J. Clin. Invest 116:940-952 (2006)) recently reported that murine C3H/10T1/2 cells which co-expressed BMP-2 and a truncated form of Smad8, but not truncated Smad1 or 5, exhibit a tenocyte morphology, suggesting a possible special role for Smad8 in tendon formation.

Recent work has demonstrated an important role for repulsive guidance molecule (RGM) co-receptor proteins in efficient BMP signaling. These co-receptors have been shown to bind both BMP-2 and BMP-12. Additionally, small-interfering (siRNA) knock-down of any of RGMa, b, or c (particularly RGMa or RGMc) decreased the in vitro response of C2C12 cells to these morphogens. Notably, TGF-β signaling was unaffected by suppression or over-expression of the RGM molecules. For additional discussion of RGMs in BMP signaling, see, for example, Halbrooks et al., J. Molec. Sig. 2:4 (2007). The NCBI GeneID numbers for some of the (human) genes believed to be necessary to respond to BMP-12-related proteins are listed in Table 4.

TABLE 4

| Molecule(s) | GeneID |
|---|---|
| ALK3, ALK6 | 657, 658 |
| BMPR2, ActR2a | 659, 11480 |
| SMAD1, 5, 8 | 4086, 4090, 4093 |
| SMAD4 | 4089 |
| RGMa, b, c | 56963, 285704, 148738 |

In addition to the molecules already discussed, efficient response to BMP-12-related proteins likely requires additional factors. For a general discussion of TGF-β family receptor signaling see, for example, Mazerbourg and Hsuch Human Reproduction Update 12:373-83 (2006).

Cells lines can be used in the methods of the invention at various passages—preferably an early passage, for example, at least passage 1, 2, 3, 4, 5, 6, 7, 8, 9, 10, 12, 14, 16, 18, 20, 25, 30, or more. In particular embodiments the cells are assayed at about passage 3, 4, 5, 6, or 7. In more particular embodiments the cells are assayed at about passage 3.

In the methods of the invention, cells are incubated with the test sample for sufficient time to allow expression of a BMP-12-related-activity-marker. Cells can be incubated with the test sample for at least about 1, 2, 5, 6, 12, 18, 24, 36, 48, 60, 72 hours; or 4, 5, 6, 7, 8, 9, 10 days, or more. In particular embodiments, the cells are incubated with the test sample for 2, 3, 4, or 5 days. In particular embodiments, the cells are incubated with the test sample for 3 days.

The cells may be seeded at any density that allows expression and detection of one or more BMP-12-related-activity-markers. In some embodiments, this may require only a single cell and amplification of the marker(s) by means well-known in the art. In other embodiments cells are seeded at a density of about 5×10³-8×10⁵ cells/well of a standard 12-well tissue culture dish (that is, about 1,316 to 210,000 cells/cm²) or more. In particular embodiments, the cells are seeded at a density of at least about 6,500; 13,000; 26,000, 52,000; 104,000; 150,000; 200,000 cells/cm²; or more.

### Expression of BMP-12-Related-Activity-Markers

Expression levels of BMP-12-related-activity-markers (nucleic acid or protein) may be detected by a variety of means well known in the art.

Nucleic acid BMP-12-related-activity-markers can be quantified by detection of mRNA transcripts by, for example, Northern blot, cDNA or oligonucleotide microarray, branched DNA, or quantitative PCR. Many methods of quantifying mRNA transcript abundance first require reverse transcription of mRNA, followed by random priming of the nascent single-stranded complementary “cDNA” for second strand synthesis to produce double stranded cDNA.

Quantifying cDNA products may be done by polymerase chain reaction (PCR, see, for example, U.S. Pat. No. 4,683,202) after amplification or during amplification, also referred to as real-time quantitative PCR. For a review of quantitative real-time PCR, see Kubista et al., Mol. Asp. Med. 27:95-125 (2006) and Bustin, J. Mol. Endocrinology. 25:169-93 (2000).

Quantitative PCR methods typically require a pair of conventional PCR primers to produce an amplicon. This amplicon can be short, for example, about 50-100 bases. In some embodiments, the amplicon is at least about 20, 30, 50, 70, 90, 100, 120, 150, 200, 300, 400, 500, or more bases. Conventional PCR primer pairs anneal to complementary strands of the target sequence, with their 3′ ends facing towards each other, so as to amplify the intervening sequence, that is, the target amplicon. Primers can be designed by means well known in the art, including visual inspection of the sequence or computer-assisted primer design. Numerous software packages are available to assist in the primer design, including DNAStar™ (DNAStar, Inc., Madison, Wis.), OLIGO 4.0 (National Biosciences, Inc.), Vector NTI® (Invitrogen), Primer Premier 5 (Premierbiosoft), and Primer3 (Whitehead Institute for Biomedical Research, Cambridge, Mass.; http://frodo.wi.mit.edu/). Primers are designed taking into account, for example, the target sequence to be amplified, specificity, length, desired melting temperature, secondary structure, primer dimers, GC content, pH and ionic strength of the buffer solution, and the polymerase enzyme.

Some quantitative PCR methods use nucleic acid dyes, which bind all nucleic acids in a mixture to estimate amplicon abundance. Nucleic acid dyes include ethidium bromide, propidium iodide, acridine orange, and cyanine dyes, such as SYBR™ Green I.

Other methods specifically detect the target amplicon with one or more probes complementary to the amplicon and which can only be detected upon annealing to the target sequence. Such methods include TaqMan™, which uses a hydrolyzable probe containing detectable reporter and quencher moieties, which are released by DNA polymerase's 5′→3′ exonuclease activity (U.S. Pat. No. 5,538,848); molecular beacon, which uses a hairpin probe with reporter and quenching moieties at opposite termini (U.S. Pat. No. 5,925,517); fluorescence resonance energy transfer (FRET) primers, which use a pair of adjacent primers with fluorescent donor and acceptor moieties, respectively (U.S. Pat. No. 6,174,670); and LightUp™, a single short probe which fluoresces only when bound to the target (U.S. Pat. No. 6,329,144). Similarly, Scorpion™ (U.S. Pat. No. 6,326,145) and SimpleProbes™ (U.S. Pat. No. 6,635,427) use single reporter/dye probes. Amplicon-detecting probes are designed according to the particular detection modality used, and as discussed in the above-referenced patents. In general, the principles discussed for conventional PCR primer pairs will be applicable to the design of amplicon-detecting primers as well.

In some embodiments, THBS4 mRNA is detected by qRT-PCR of a particular exon or exon boundary, for example, an exon corresponding to human or mouse exon 1, 2, 3, 4, 5, 6, 7, 8, 9, 10, 11, 12, 13, 14, 15, 16, 17, 18, 19, 20, 21, 22, or boundaries therebetween. By corresponding to, it is meant of an analogous position between two or more sequences. For example, the sequence of RNA molecule A “corresponding to exon X of RNA molecule B” refers to the analogous sequence in RNA molecule A, as determined by means well known in the art, for example, sequence alignment, BLAST, CLUSTALW, and Smith-Waterman. In particular embodiments, the exon corresponds to a mouse exon, for example, the exon 1-2 boundary, exon 3, the exon 6-7 boundary, the exon 10-11 boundary, the exon 15-16 boundary, or the exon 18-19 boundary. In more particular embodiments the exon is exon 3 or the exon 6-7 boundary. In yet more particular embodiments, the exon is exon 3.

In other embodiments, the BMP-12-related-activity-marker is a protein, or a fragment thereof. Protein or peptide BMP-12-related-activity-markers of the invention can be detected by any technique known in the art, including, Western blot, ELISA, mass spectrometry, chromatography, and immunochemistry. For a discussion of immunological techniques, including, ELISA and Western blotting, see David Wild (ed.) The Immunoassay Handbook Elsevier Science; 3rd edition (2005). Many methods of quantifying protein levels rely on antibodies. The term “antibody,” as used herein, refers to an immunoglobulin or a part thereof, and encompasses any polypeptide comprising an antigen-binding site regardless of the source, species of origin, method of production, and characteristics. As a non-limiting example, the term “antibody” includes human, orangutan, mouse, rat, goat, sheep, and chicken antibodies. The term includes but is not limited to polyclonal, monoclonal, monospecific, polyspecific, non-specific, humanized, camelized, single-chain, chimeric, synthetic, recombinant, hybrid, mutated, and CDR-grafted antibodies. For the purposes of the present invention, it also includes, unless otherwise stated, antibody fragments such as Fab, F(ab′)2, Fv, scFv, Fd, dAb, VHH (also referred to as nanobodies), and other antibody fragments that retain the antigen-binding function. Antibodies also refers to antigen-binding molecules that are not based on immunoglobulins, as further described below.

Antibodies can be made, for example, via traditional hybridoma techniques (Kohler and Milstein, Nature 256: 495-499 (1975)), recombinant DNA methods (U.S. Pat. No. 4,816,567), or phage display techniques using antibody libraries (Clackson et al., Nature 352: 624-628 (1991); Marks et al., J. Mol. Biol. 222: 581-597 (1991)). For various other antibody production techniques, see Antibodies: A Laboratory Manual, eds. Harlow et al., Cold Spring Harbor Laboratory, 1988.

The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is responsible for specific interactions with the antigen-binding domain of an antibody. An antigen-binding domain may be provided by one or more antibody variable domains (e.g., a so-called Fd antibody fragment consisting of a VH domain). An antigen-binding domain can comprise an antibody light chain variable region (VL) and an antibody heavy chain variable region (VH). Antibodies from camels and llamas (Camelidae, camelids) include a unique kind of antibody, which is formed by heavy chains only and is devoid of light chains. The antigen-binding site of such antibodies is one single domain, referred to as VHH. These have been termed “camelized antibodies” or “nanobodies”. See e.g. U.S. Pat. Nos. 5,800,988 and 6,005,079 and International Application Publication Nos. WO 94/04678 and WO 94/25591, which are incorporated herein by reference.

The term “repertoire” refers to a genetically diverse collection of nucleotides, e.g., DNA, sequences derived wholly or partially from sequences that encode expressed immunoglobulins. The sequences are generated by in vivo rearrangement of, e.g., V, D, and J segments for H chains and, e.g., V and J segment for L chains. Alternatively, the sequences may be generated from a cell line by in vitro stimulation and in response to which rearrangement occurs. Alternatively, part or all of the sequences may be obtained by combining, e.g., unrearranged V segments with D and J segments, by nucleotide synthesis, randomized mutagenesis, and other methods as disclosed in U.S. Pat. No. 5,565,332.

In some embodiments, the term “antibody” includes an antigen-binding molecule based on a scaffold other than an immunoglobulin. For example, non-immunoglobulin scaffolds known in the art include small modular immunopharmaceuticals (see, e.g., U.S. Patent Application Publication Nos. 20080181892 and 20080227958 published Jul. 31, 2008 and Sep. 18, 2008, respectively), tetranectins, fibronectin domains (e.g., AdNectins, see U.S. Patent Application Publication No. 20070082365, published Apr. 12, 2007), protein A, lipocalins (see, e.g., U.S. Pat. No. 7,118,915), ankyrin repeats, and thioredoxin. Molecules based on non-immunoglobulin scaffolds are generally produced by in vitro selection of libraries by phage display (see, e.g., Hoogenboom, Method Mol. Biol. 178:1-37 (2002)), ribosome display (see, e.g., Hanes et al., FEBS Lett. 450:105-110 (1999) and He and Taussig, J. Immunol. Methods 297:73-82 (2005)), or other techniques known in the art (see also Binz et al., Nat. Biotech. 23:1257-68 (2005); Rothe et al., FASEB J. 20:1599-1610 (2006); and U.S. Pat. Nos. 7,270,950; 6,518,018; and 6,281,344) to identify high-affinity binding sequences.

The term “specific interaction,” or “specifically binds,” or the like, means that two molecules form a complex that is relatively stable under physiologic conditions. Specific binding is characterized by a high affinity and a low to moderate capacity. Nonspecific binding usually has a low affinity with a moderate to high capacity. Typically, the binding is considered specific when the affinity constant Ka is higher than 10⁶ M⁻¹, or preferably higher than 10⁸ M⁻¹. In some embodiments, antibodies for use in the methods of the invention bind their antigen(s) with association constants of at least 10⁶, 10⁷, 10⁸, 10⁹ M⁻¹, or higher. If necessary, non-specific binding can be reduced without substantially affecting specific binding by varying the binding conditions. Such conditions are known in the art, and a skilled artisan using routine techniques can select appropriate conditions. The conditions are usually defined in terms of concentration of antibodies, ionic strength of the solution, temperature, time allowed for binding, concentration of non-related molecules (e.g., serum albumin, milk casein), etc.

Antibodies to a peptide BMP-12-related-activity-marker may be any of the varieties discussed above. They may be commercially available or produced by routine methods known in the art, as described above. For example, THBS4 antibodies are commercially available from R&D Systems (catalog numbers AF2390 and BAF2390 are polyclonal anti-human antibodies; and catalog number MAB2390 is a monoclonal anti-human antibody) and Santa Cruz Biotechnology (catalog numbers sc-28293 and sc-55464 are monoclonal anti-human antibodies; and catalog numbers sc-20646 and sc-7657 are polyclonal anti-human antibodies). Alternatively, a peptide sequence of a BMP-12-related-activity-marker—such as THBS4, given in SEQ ID NOs: 19 (human) or 21 (mouse)—can be used to design or screen antibodies that contain immunoglobulin-based or non-immunoglobulin-based scaffolds.

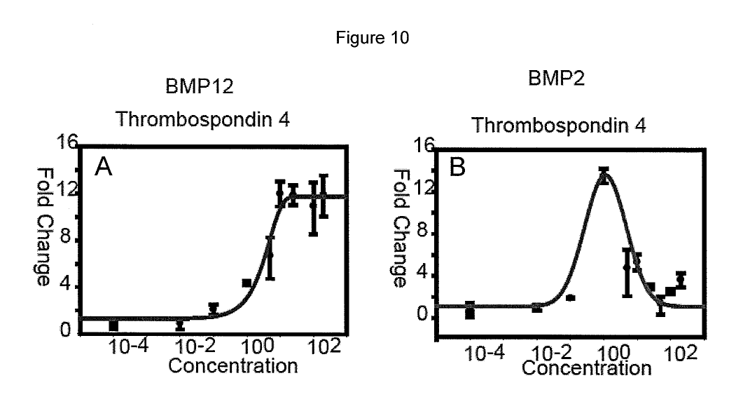

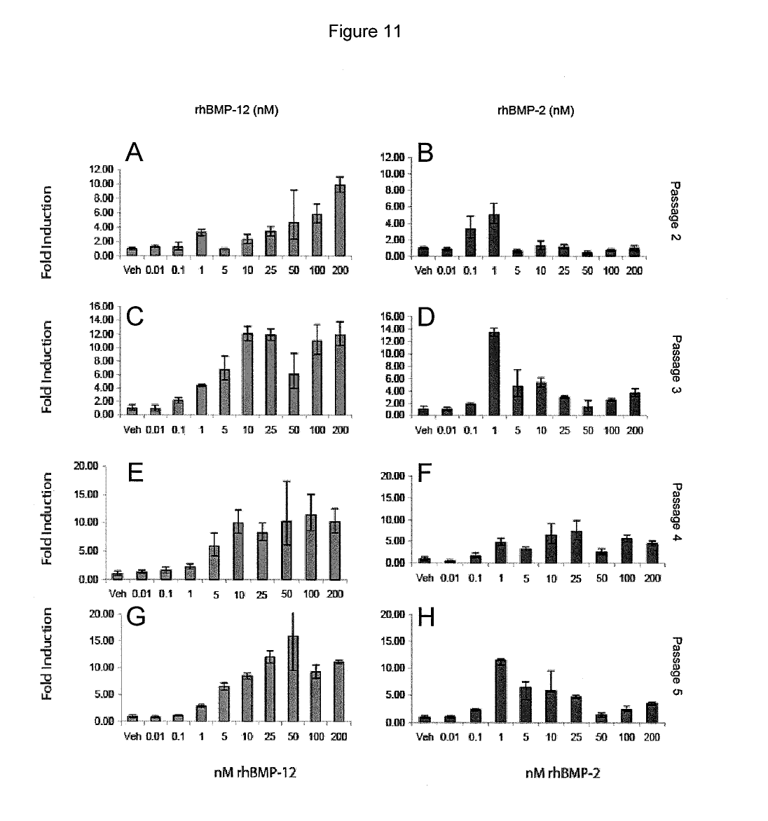

In some embodiments, a dose-responsive increase corresponds to, for example, a sigmoidal response; a saturable increasing response; a saturable monotonically increasing response; and equivalent terminology readily apparent to the skilled artisan. By saturable, it is meant that increasing concentration of an independent variable eventually no longer produces an increase in the dependent variable, whose levels then plateau. A non-limiting example includes a plot of the fold-induction of THBS4 mRNA expression versus BMP-12 concentration, which shows a characteristic s-shaped curve, as seen in, for example, FIGS. 10A and 11A-11C. THBS4 mRNA levels increase in response to increasing BMP-12 concentration and then plateau, such that increasing BMP-12 concentration no longer elicits a significant increase in THBS4 mRNA levels. Such curves are common in biology, for example, the fractional O₂ saturation of hemoglobin, described by Archibald Hill. Sigmoid curves themselves are a special case of the logistic function. Another example of a saturable increasing function in biology is the plot of enzyme-mediated reaction velocity against increasing substrate concentration, which plateaus at the Vₘₐₓ asymptote.

### Other Embodiments

The invention provides methods for detecting and/or measuring the BMP-12-related protein activity in a test sample. In one embodiment, the method includes the steps of contacting a cell with the test sample, detecting the level of a BMP-12-related-activity-marker, and detecting the level of an osteogenic-marker. The ratio of the levels of the BMP-12-related-activity-marker and osteogenic-marker indicates presence of BMP-12-related protein activity in the test sample. In particular embodiments, the osteogenic-marker is, for example, osteocalcin, or alkaline phosphatase. In some embodiments, for a BMP-12-related protein activity is, for example, greater than 1, positive (log ratio), dose-dependent increasing, not less than 1, non-negative (log ratio), or not dose-dependent decreasing. In certain embodiments, the ratio of BMP-12-related-activity-marker and osteogenic-marker, such as the THBS4/OST mRNA ratio, is probative of the specificity of a BMP-12-related protein activity in a test sample.

The invention also provides methods of testing the effect of a test compound on a compound with BMP-12-related protein activity. The methods require a first test sample containing a compound known to have BMP-12-related protein activity and a second test sample containing the test compound and the compound known to have BMP-12-related protein activity. BMP-12-related protein activity of the two test samples is detected and/or measured by the methods of the invention as described in detail above. Comparison of the BMP-12-related protein activity of the test samples with and without the test compound indicates the effect of the test compound on the activity of the compound with BMP-12-related protein activity. In some embodiments, the test compound may be, for example, a protein, peptide, polypeptide, antibody, or pharmaceutical. In some embodiments, the methods of the invention can be used to detect and/or measure the effect of a test compound which may enhance, amplify, attenuate, diminish or block the activity of the compound with BMP-12-related protein activity. In particular embodiments, the methods of the invention can be used to detect and/or measure the effect of a test compound which may increase or decrease the activity of the compound with BMP-12-related protein activity by at least, for example, 30, 50, 70, 90, 99%; or 1, 2, 4, 10, 25, 50, 100, 250, 500, 1000-fold, or more. In more particular embodiments, the compound with BMP-12-related protein activity is a BMP-12-related protein.

For all patents, applications, GeneIDs, Reference Sequences, or other reference cited herein, it should be understood that it is incorporated by reference in its entirety for all purposes as well as for the proposition that is recited. Where any conflict exists between a document incorporated by reference and the present application, this application will dominate.

### EXAMPLES

Reference will now be made in detail to the present exemplary embodiments of the invention, examples of which are illustrated in the accompanying drawings.

### Example 1

### Identification of Tendon Selective Genes

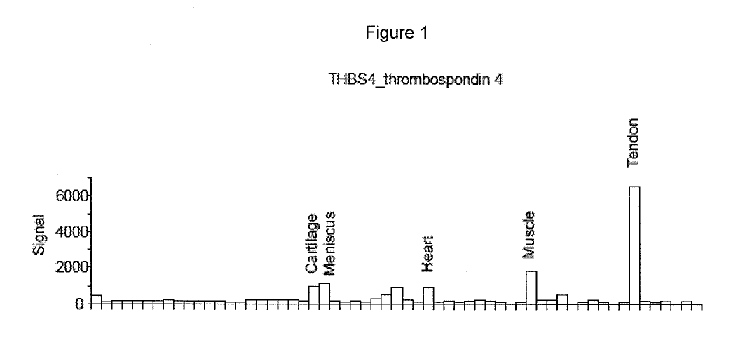

Tendon, paratenon, ligament, meniscus, muscle, adipose tissue, cartilage, bone and bone marrow were harvested from 10 male, 18 week-old Sprague-Dawley rats. Human tendon, meniscus, cartilage, muscle, bone marrow, bone, and adipose tissue were obtained from cadaver donors (NDRI). Transcriptional profiling was performed using Affymetrix rat genome 230 2.0 arrays (30,000 transcripts) or Affymetrix human genome U133 2.0 Plus arrays (54,000 transcripts). Signal values were determined using Gene Chip Operating System 1.0 (GCOS, Affymetrix). A gene was considered detectable if the mean expression in any tissue was greater than 100 signal units and the percentage of samples with a present call was greater than or equal to 66%. A gene was considered differentially expressed if the p-value based on an ANOVA test was <0.0005 and the difference between tissues was at least four-fold. A gene was considered tissue selective if its expression was 2-fold higher in a specific tissue than in all of the eight other tissues. Genes identified as tissue selective in the musclosketal tissues were then screened against an internal database of 110 human tissues and 40 rat tissues. Genes that were expressed 2-fold higher in tendon versus all other tissues were identified. The results of this analysis revealed one of the tendon/ligament selective genes, thrombospondin-4 (THBS4) was highly expressed in tendons and ligaments and only minimally expressed in cardiac muscle. See FIG. 1. Additional genes with tendon-specific expression included Tenomodulin (TNMD, Homo sapiens GeneID: 64102), and CCDC3 (Homo sapiens GeneID: 83643).

### Example 2

### Identification of Genes with Expression Patterns Similar to THBS4

C3H/10T1/2 cells were treated with 0.1, 1.0, 10, or 100 nM rhBMP-2, rhBMP-12, or rhBMP-13 for three days. RNA was isolated and subjected to microarray analysis. Data was imported in Expressionists™ (GeneData™) for statistical analysis. Differentially regulated genes were identified using a 2-way ANOVA analysis with treatment and treatment amount as the factors and treatment as the effect. Genes with an ANOVA p<0.0001 were grouped by self organizing maps (SOM) algorithm into 3×5 grid. SOM clusters representing the “THBS4” pattern were identified. This led to identification of ˜40 qualifiers showing the differences in regulation following treatment with rhBMP-12 or BMP-13; and rhBMP-2. The GeneIDs and mRNA reference sequences for the genes corresponding to these identifiers are shown in Table 2.

### Example 3.1

### Cell Culture, RNA Isolation, and cDNA Synthesis

Standard aseptic cell culture techniques were used. Cells were maintained at 37° C. and 5% CO₂ unless indicated otherwise. All media were sterile filtered. Murine C3H/10T1/2 clone 8 mesenchymal stem cells were purchased from ATCC (Cat # CCL-226). Cells were cultured in 1× Minimum Essential Media (MEM; Cellgro Cat # 10-010-CV) supplemented with 10% inactivated fetal bovine serum (FBS; Invitrogen Cat # 10082-142) without the addition of antibiotics. Exponentially growing cells (2×10⁵ cells at a density of 2×10⁵ cells/ml) from passage 2-16 were cultured in clear flat bottom 12 well plates (Becton Dickinson Cat # 353225) overnight. The following day, the media was replaced with MEM supplemented with 1% FBS without antibiotics. A single dose of each rhBMP was added to the culture and the cells were grown at 37° C. for 3 days. Cells were harvested and RNA was isolated using the RNeasy kit (Qiagen Cat # 74106). 250 ng of purified RNA was used to synthesize cDNA using the Superscript™ cDNA kit (Invitrogen; Cat # 18080-400) with 5 nM oligo dT and 5 ng/ml random hexamer primers in 10 μl. 3 μl of each cDNA reaction was used for each TaqMan™ (Applied Biosystems) real-time PCR.

### Example 3.2

### Real-time PCR Analyses

Real-time RT-PCR analysis was carried out using FAM-labeled fluorogenic LUX™ primers from ABI. Quantitative real-time PCR reactions (qRT-PCR) were conducted using TaqMan™ Pre-Developed Assay Reagents and 10× buffer (Applied Biosystems). The reagent was diluted to the appropriate working concentration in a final volume of 25 μl. The reaction mixture was as follows: PDAR (TaqMan™ Pre-Developed Assay Reagents) 1.25 μl, Reverse transcriptase 0.125 μl, DEPC water 8.125 μl, cDNA 3 μl.

PCR reactions were run on an automated fluorometer (ABI Prism 7900 Sequence Detection System, Applied Biosystems) in a 96-well format. PCR conditions for all reactions were as follows: 1 cycle of 2 mins at 50° C. and 10 mins at 95° C.; and 40 cycles of 15 secs at 95° C. and 1 min at 60° C. Relative expression was determined by using the C_{T} (Cycle threshold) method using Sequence Detector Software version 2.2.2. cDNA samples of each biological timepoint were evaluated in triplicate with the primer pair for each gene. Unless indicated otherwise, results were normalized to GUSB expression and expressed as a ratio of expression of each gene in each rhBMP treated sample compared to the expression of that gene in the control untreated sample, that is, the ΔΔCt method.

### Example 3.3

### Statistical Analysis

The common ΔΔCt method was used in all analyses. Briefly, raw Ct values were obtained from the Sequence Detection System (SDS) software v2.3 supplied by ABI. The Ct value is the cycle number at which the fluorescence generated within a reaction crosses a threshold within the linear portion of an amplification curve. Thus, a well's Ct value is the amplification cycle at which a sufficient number of amplicons accumulated to be at a statistically significant point above the baseline. Typically this is about 10 times the standard deviation of the machine baseline. The ΔCt value was determined by subtracting the average GUSB Ctvalue from the average THBS4 Ctvalue (that is, ΔCt_{THBS4}=<Ct_{THBS4}>-<Ct_{GUSB}>; read, <X> as the average value of X). The standard deviation of the difference is calculated from the standard deviations of the THBS4 and GUSB. The standard deviation of the difference is calculated from the standard deviations of the THBS4 and GUSB values using the following formula:

S_{ΔCt}=\sqrt{S_{Thbs4}²+S_{GUSB}²}

The basic ΔΔC_{T} calculation is ΔΔCt=(ΔCt_{THBS4})ₜᵣₑₐₜₘₑₙₜ−(ΔCt_{THBS4})_{no treatment}, for example, (ΔCt_{THBS4})BMP-12-(ΔCt_{THBS4})vehicle. The standard deviation of ΔC_{T} is the same as the standard deviation of the ΔCtvalue. The range given for THBS4 in rhBMP treated samples relative to untreated samples is determined by evaluating the expression: 2^{ΔCt}.

### Example 3.4

### THBS4 Tagman Probe Optimization

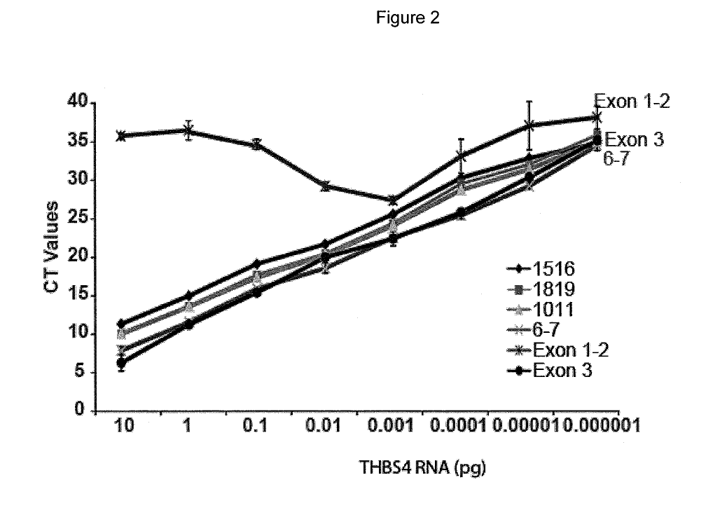

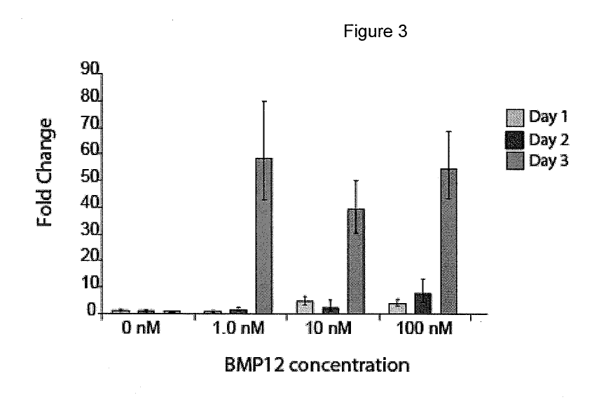

Five different primer/probes at defined intervals of the THBS4 transcript (Table 5) were designed. These were tested along with a probe available as an Assay-on-Demand that spanned the exon 1-2 boundary. The mouse THBS4 transcript is 3160 bp and contains 22 exons. THBS4 mRNA was generated by in-vitro transcription for use as a template to determine the linearity, sensitivity and reproducibility of each primer/probe. Eight concentrations ranging from 10 pg to 0.0000001 pg of THBS4 transcript were used in each RTPCR reaction. FIG. 1 shows the Ct values generated for each of 6 TaqMan™ probe sets across the THBS4 RNA concentration range. As shown in FIG. 2, most probes had a linear response and most had similar sensitivity. The exon 1-2 probe did not show a linear response throughout the entire RNA range, but was linear through the Cts at which THBS4 RNA is measured in C3H/10T1/2 cells (0.001 pg to 0.000001 pg). This probe was used for initial experiments (FIGS. 2 and 3) since it was available off the shelf.

In FIG. 2, probes corresponding to exon 3 and the exon 6-7 boundary show the lowest Ct values at each amount of THBS4 RNA. We chose the exon 3 probe set for future use since it showed good sensitivity, was robust, and linear. The strong R² value for this probe set was 0.9947, suggesting that it would be suitable for future experiments analyzing THBS4 mRNA induction by rhBMP-12. Table 5 contains the AssayID, exon, and sequences of the TaqMan™ probes tested.

TABLE 5

| | | | |
|---|---|---|---|
| Assay ID | Exon | Primer | Sequence, 5′→3′ |

| | | | | |
|---|---|---|---|---|
| NM_011582-6E7 | 6-7 | Forward | TTTGCGAAACACCATTGCTGAAT | (SEQ ID NO:1) |
| | | Reverse | GCACCAGTGTGTTTGGAGTTG | (SEQ ID NO:2) |
| | | Reporter | CCAAGCTTGTGGTCCTC | (SEQ ID NO:3) |
| NM_011582-1011 | 10-11 | Forward | GCATGCGCAGTGCATTGA | (SEQ ID NO:4) |
| | | Reverse | CCACATCCTTTCCACAGACATAGC | (SEQ ID NO:5) |
| | | Reporter | CACCACACACACACGTC | (SEQ ID NO:6) |
| NM_011582-1516 | 15-16 | Forward | GACTCCTGTGACACCAACCA | (SEQ ID NO:7) |
| | | Reverse | GCTGGGAGCTGTTAATGACTGT | (SEQ ID NO:8) |
| | | Reporter | CATCCCCGTCACTGTCT | (SEQ ID NO:9) |
| NM_011582-1819 | 18-19 | Forward | GCTCAGATCGACCCCAACTG | (SEQ ID NO:10) |
| | | Reverse | TCCCTTCAAAGTCAACTCCGTTAAA | (SEQ ID NO:11) |
| | | Reporter | CCGTGTACCCAACAGC | (SEQ ID NO:12) |
| Mm00449057_m1 | 1-2 | Forward | ABI catalog number Mm00446953-m1 | |
| | | Reverse | ABI catalog number Mm00446953-m1 | |
| | | Reporter | ABI catalog number Mm00446953-m1 | |
| MTHBSPUSH-USH4 | 3 | Forward | ACTCGGTGCGCAACCT | (SEQ ID NO:13) |
| | | Reverse | CAATTCAATGGACTCTGGGTTCTG | (SEQ ID NO:14) |
| | | Reporter | CCCCAGAGCCTTTTCT | (SEQ ID NO:15) |

### Example 4

### Optimization of C3H/10T1/2 Treatment Conditions for THBS4 mRNA Induction by rhBMP-12

Multiple experimental conditions were optimized for induction of THBS4 mRNA by rhBMP-12. These included BMP dose, incubation time, cell density, and well size. Additionally, C3H/10T1/2 cells were tested at multiple passages.

### Example 4.1

### Optimization of Time for Maximal Induction of rhBMP-12 Induced THBS4

Initial experiments (utilizing the exon 1-2 probe, the only commercially available probe) determined that maximal induction of THBS4 mRNA occurred following 3 days of rhBMP-12 treatment with little to no induction at 1 or 2 days. At doses of 1, 10, and 100 nM, rhBMP-12 produced a robust induction of THBS4 mRNA (FIG. 3). Initial experiments used GAPDH as a “housekeeping” gene to normalize to. GAPDH levels, however, were found to change during different treatment regimens, thus GAPDH is not an ideal normalizing transcript. Subsequent experiments showed that the level of GUSB mRNA remains relatively constant. GUSB was used as a control for all other experiments. Commercially available GUSB TAQMAN™ probes were used (ABI catalog number Mm00446953_m1). This probe set corresponds to the exon 1-2 boundary of the mouse reference sequence NM_{—}010368.1, which is incorporated by reference in its entirety.

Assay conditions for the induction of THBS4 mRNA following 3 days of rhBMP-12 treatment were subsequently optimized.

### Example 4.2

### Optimization of Well Size

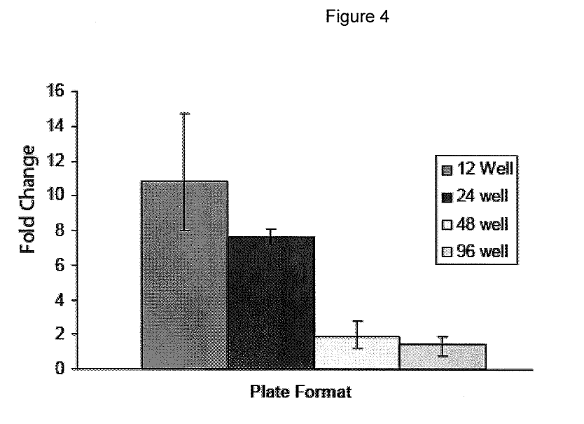

Initial studies examining the induction of THBS4 by rhBMP-12 in C3H/10T1/2 cells were performed in 12 well culture dishes. In order to increase the throughput, we evaluated the use of smaller well formats. Cells at the same equivalent density (2×10⁵ (cells/well) 3.8 cm²/well=5.26×10⁴ cells/cm²) were plated in 12, 24, 48 or 96 well and treated with 100 nM rhBMP-12 for three days. THBS4 induction was monitored by TaqMan™ qRT-PCR using the exon 3 probes. Results are shown in FIG. 4. There was a reduction in the magnitude of THBS4 induction when cells were grown in smaller formats such as 48 and 96 well plates. Use of 12 well plates allowed increased mRNA yield, allowing for testing of additional transcripts. Thus, the 12 well plate format was chosen for further optimization.

### Example 4.3

### Optimization of Cell Density

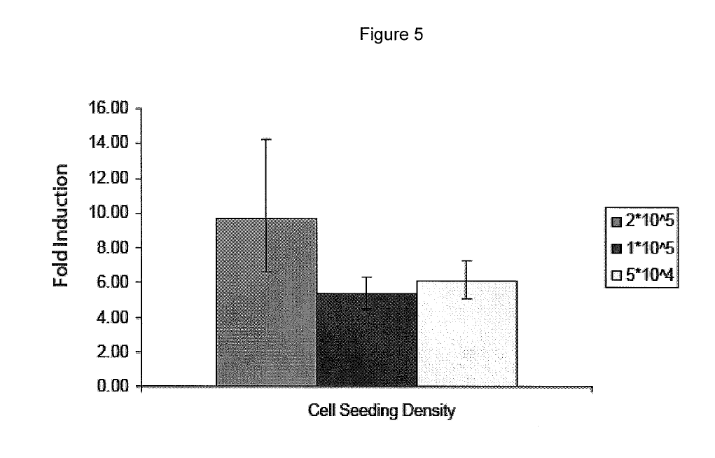

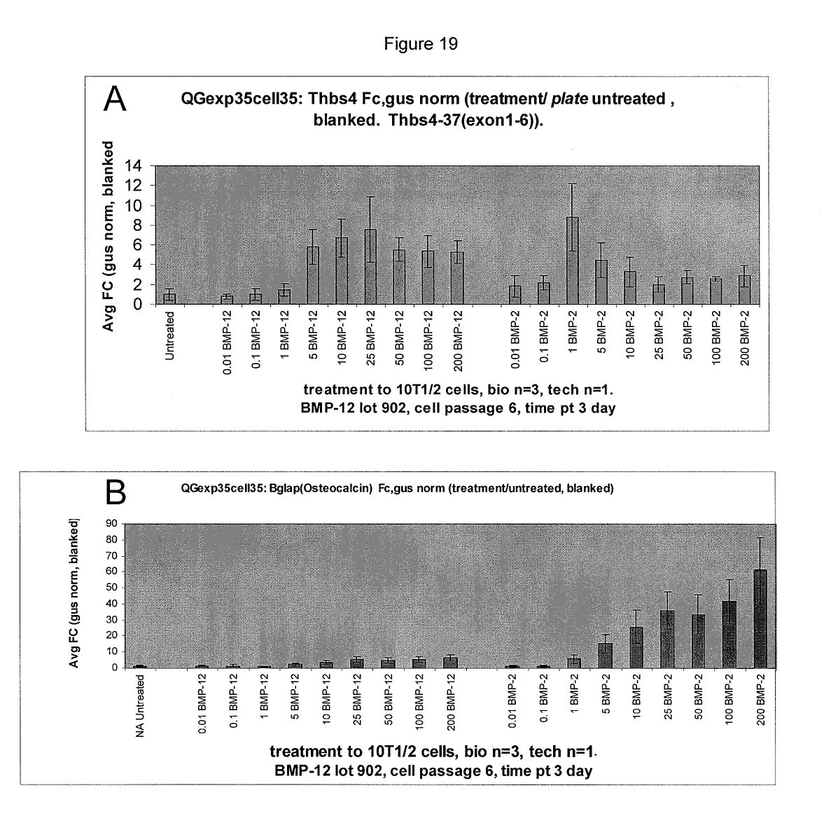

Using the 12 well plate format, four different cell densities ranging from 1×10⁴ cells to 2×10⁵ cells per well were tested. FIG. 5 shows representative experiments at 5×10⁴, 1×10⁵, and 2×10⁵ cells/well (1×10⁴ cells did not generate enough RNA for analysis using standard purification techniques). The induction of THBS4 mRNA by rhBMP-12 at 100 nM was higher when using the highest density of cells: 2×10⁵ cells/well. Comparable data was obtained in independent experiments where THBS4 mRNA levels were monitored using a branched DNA assay (FIG. 19).

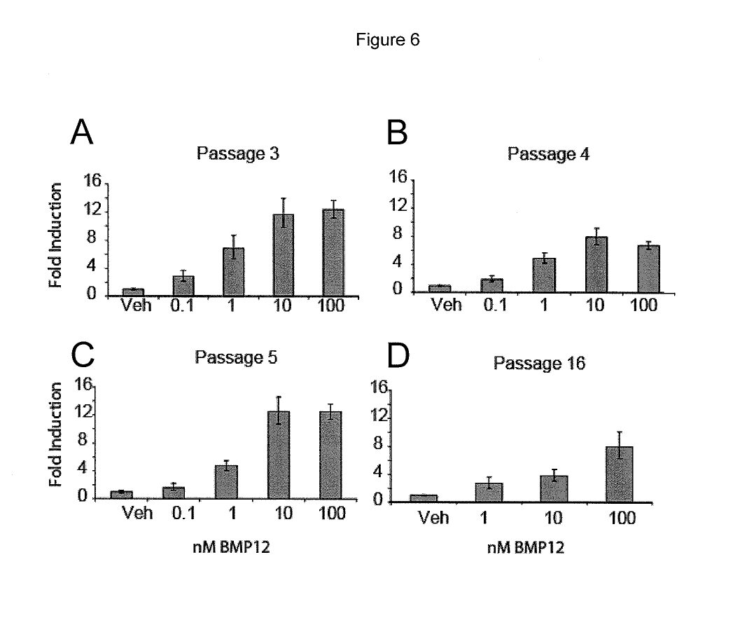

Next, a cell passage window for consistent response to rhBMP-12 was evaluated. A passage is considered a trypsinization of confluent cells followed by re-plating. Cells thawed from the ATCC stock were considered passage 0. rhBMP-12 dependent dose curves (0.1 nM to 100 nM) were generated with cells plated at 2×10⁵ cells/well of a 12 well plate, treated for 3 days in MEM+1% FCS and rhBMP-12. Passage 16 cells obtained by thawing a stock of passage 10 cells and passaging the cells 6 times. As shown in FIG. 6, rhBMP-12 induced expression of THBS4 in a dose dependent manner from 1 nM to 100 nM. All passages tested showed a similar dose dependent induction and maximum magnitude of induction (8-12 fold). Additional cell passages beyond passage 16 were not tested.

### Example 4.4

### Fully Oxidized BMP12 does not Induce THBS4

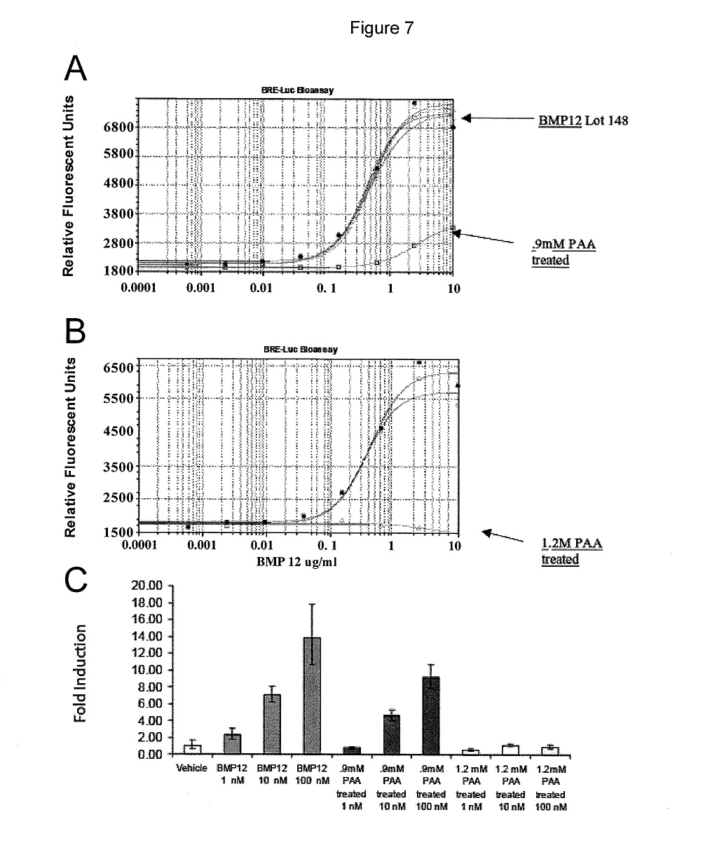

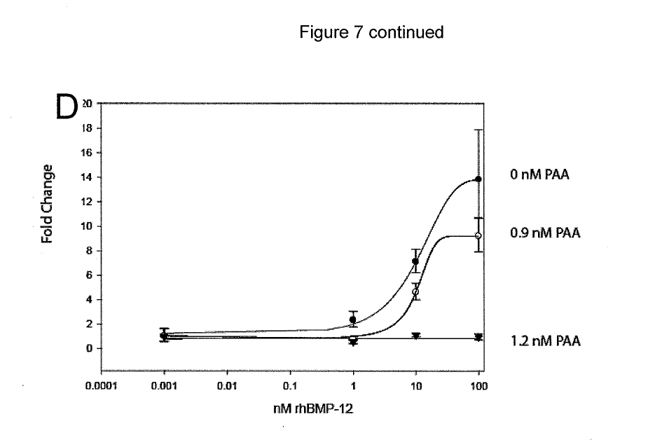

BMP12 was incubated with either 0.9 mM or 1.2 mM peracetic acid (PAA) for 2 hours to deactivate the protein through oxidation. The 1.2 mM PAA treated rhBMP-12 was completely inactive in the BRE-luc assay and was unable to induce THBS4 mRNA in C3H/10T1/2 cells. See FIGS. 7B, 7C, and 7D. The 0.9 mM PAA treated rhBMP-12 retained some activity in the BRE-luc and THBS4 assays. See FIGS. 7A, 7C, and 7D. In the BRE-luc assay, the 0.9 mM treated rhBMP-12 lost about 75% of its activity at 10 μg/ml, while in the THBS4 assay it lost about 40% of its activity at 100 nM (2.8 μg/ml). 100,000 cells per well of 96 well plate carrying the reporter were treated for 24 hours. Cells were treated at passage 5 in this assay. These results demonstrate that fully-oxidized BMP-12 does not induce THBS4. Accordingly, the assay is useful for detecting biologically active BMP-12-related proteins.

### Example 4.5

### Optimized Dose Response

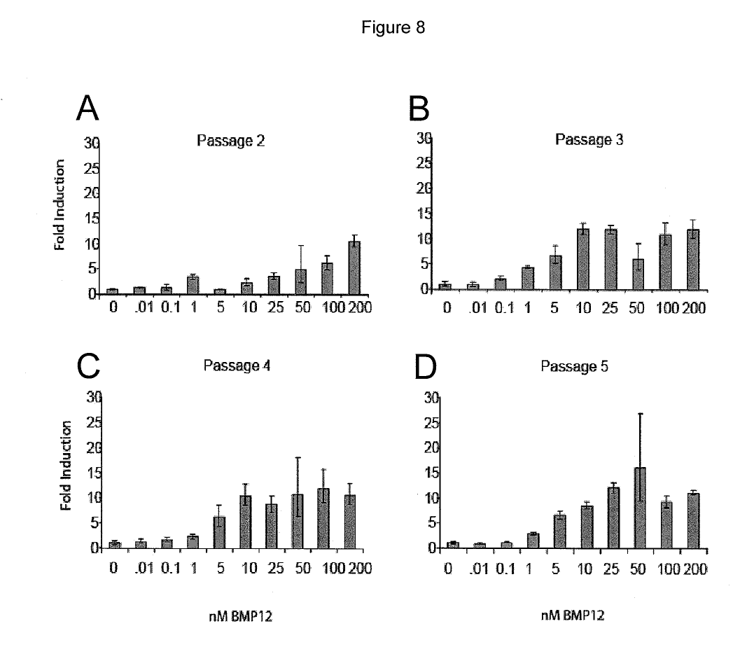

After conditions for THBS4 induction and detection were optimized, an 8 point dose curve of THBS4 mRNA using rhBMP-12 concentrations from 0.1 nM to 200 nM was determined for cells at passage 2, 3, 4, and 5. Cells were grown in 12 well plates and each plate contained 3 replicates of 3 treatments. Three wells of untreated cells were used as control on each plate. THBS4 induction was detected at 1 nM rhBMP-12, with maximal induction after 10-100 nM. See FIG. 8. These experiments showed a reproducible (10-15 fold) THBS4 induction by rhBMP-12 in cells from passage 2 up to passage 5. This is comparable to the induction seen in FIG. 6. Cells beyond passage 5 were not tested.

### Example 5.1

### BMP-2 Effect on THBS4 mRNA Expression

A bioassay for BMP-12-related protein activity of compounds such as BMP-12-related proteins should measure a tendon-related process quickly, take place in an easily manipulated cell line, and be specific to tendon-inducing activity, for example, rhBMP-12. To address signaling specificity, the activity of rhBMP-2 was tested in the methods of the invention.

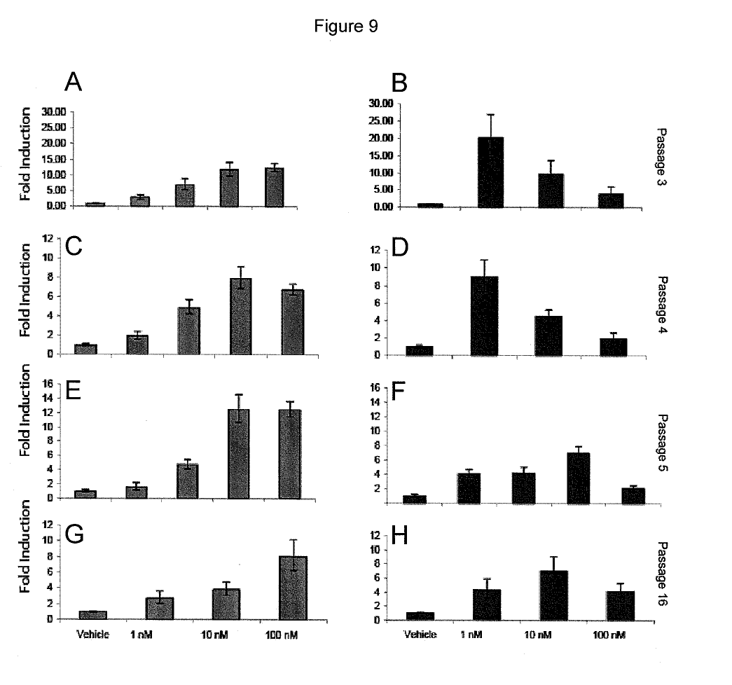

THBS4 mRNA exhibited a distinctly different dose response to rhBMP-2, compared to rhBMP-12. While the activity of rhBMP-12 followed a dose dependent increase (for example, a sigmoidal dose-response), the activity of rhBMP-2 showed a peak response curve. See FIGS. 9, 10, and 11. rhBMP-2 produced a peak induction at a 1 nM, then returned to near baseline levels at 100 nM. FIG. 9 shows the effect of rhBMP-2, which was tested concurrently with rhBMP-12 as shown in FIG. 6. In FIG. 9, THBS4 induction plateaued at a concentration of about 10 nM rhBMP-12 (FIGS. 9A, 9C, 9E, and 9G), while for rhBMP-2 (FIGS. 9B, 9D, 9F, 9H), most passages showed peak induction at 1 nM. Cells from passage 5 and 16 showed peak induction at 10 nM of rhBMP-2 but at higher doses the response was diminished, consistent with other passages.

FIGS. 10a and 10b are fitted plots of 8 point dose response curves of THBS4 mRNA in C3H/10T1/2 cells at passage 3 treated with rhBMP-12 (FIG. 10a) or rhBMP-2 (FIG. 10b) at concentrations from 0.1 nM to 200 nM. Cells were grown in 12 well plates and each plate contained 3 replicates of 3 treatments. Three wells of untreated cells were used as control on each plate.

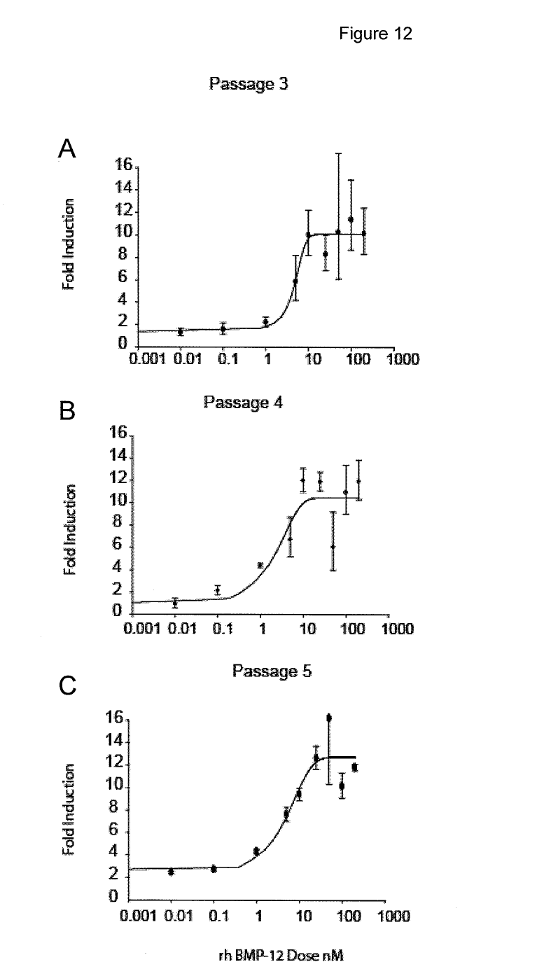

Ten point dose response curves were performed (FIG. 11) to further characterize the dose response of THBS4 mRNA to both rhBMP-12 and rhBMP-2. FIG. 11 illustrates that THBS4 mRNA levels are maximal and plateaued beginning at 10 nM of rhBMP-12. FIG. 12 provides a sigmoidal curve fit to this data. Conversely, rhBMP-2 induced maximal THBS4 expression at 1 nM and decreased to near baseline levels at 100 nM rhBMP-2. These experiments demonstrate that rhBMP-12 and rhBMP-2 induce dramatically different patterns of THBS4 expression.

### Example 5.2

### Induction of Osteocalcin mRNA

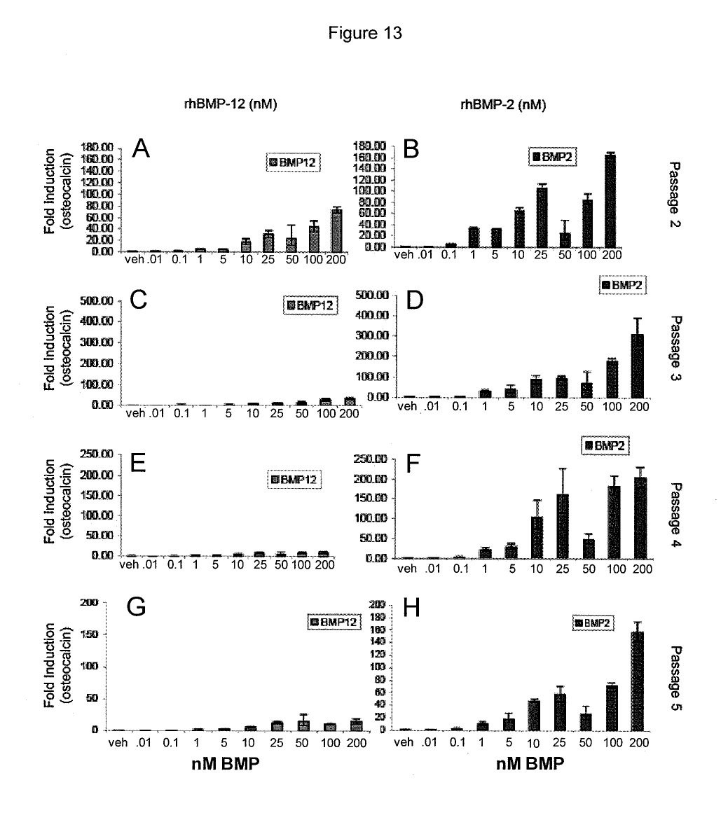

rhBMP-2 has been characterized as having strong osteogenic activity, while rhBMP-12 has minimal osteogenic activity at low doses. We therefore evaluated the ability of rhBMP-12 and rhBMP-2 to activate transcription of a known osteogenic-signaling marker osteocalcin (OST). The following primers were used to detect osteocalcin: forward 5′-CGGCCCTGAGTCTGACAAA-3′ (SEQ ID NO:16); reverse 5′-GCCGGAGTCTGTTCACTACCTT-3′ (SEQ ID NO:17); probe 5′-CCTTCATGTCCAAGCAGGAGGGCA-3′ (SEQ ID NO:18). rhBMP-2 elicited strong induction of OST mRNA (>100 fold) in a dose dependent manner (FIG. 13). In comparison, rhBMP-12 exhibited a much-reduced induction of OST (<10 fold). At high doses of rhBMP-12, there was clear up-regulation of OST, but the levels were <10 fold the maximal induced levels in rhBMP-2 treated cells at all passages tested except for passage 2 (see description below).

FIG. 13 shows osteocalcin mRNA levels for the experiments described in FIG. 11. After passage 3, rhBMP-12 elicited more than 10-fold lower induction of OST mRNA, relative to equivalent BMP-2 dose. In passage 2 cells, rhBMP-12 showed a much stronger activation of OST compared to later passage cells. However, at all passages tested, rhBMP-2 induced higher OST mRNA levels than rhBMP-12.

### Example 5.3

### THBS4/Osteocalcin Ratio as a Protein Characterization Measure

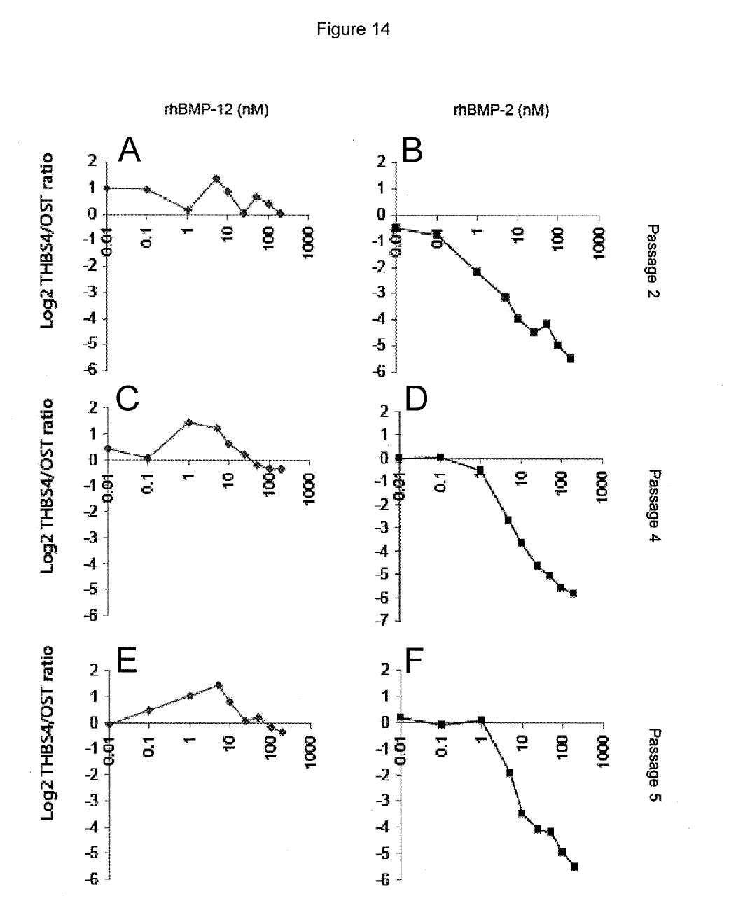

Given the profile of an increased BMP-12-related-activity-marker (THBS4) and little induction of an osteogenic signaling marker (OST), in BMP-12-treated cells, we investigated whether a ratio of THBS4 induction to OST induction could characterize the activity of rhBMP-12 in a biologically relevant manner. FIG. 14 shows the THBS4/OST ratio, log₂ transformed, for the data presented in FIGS. 11 and 13. For most cell passages tested, there was a dose dependent increase in the THBS4/OST ratio in rhBMP-12 treated samples up to the 5 nM dose. At higher doses, rhBMP-12 induced expression of osteocalcin (see FIG. 13) and therefore the ratio of THBS4 to OST began to decline back to 1. In contrast, the THBS4/OST ratio for rhBMP-2 treatments showed a dose dependent decrease, the ratio never reached above 0, and decreased sharply at higher doses. Accordingly, measurement of THBS4 mRNA induction by rhBMP12 was sufficient to characterize rhBMP-12 activity for cells at passage 3-5.

### Example 6

### Later Passage Cells

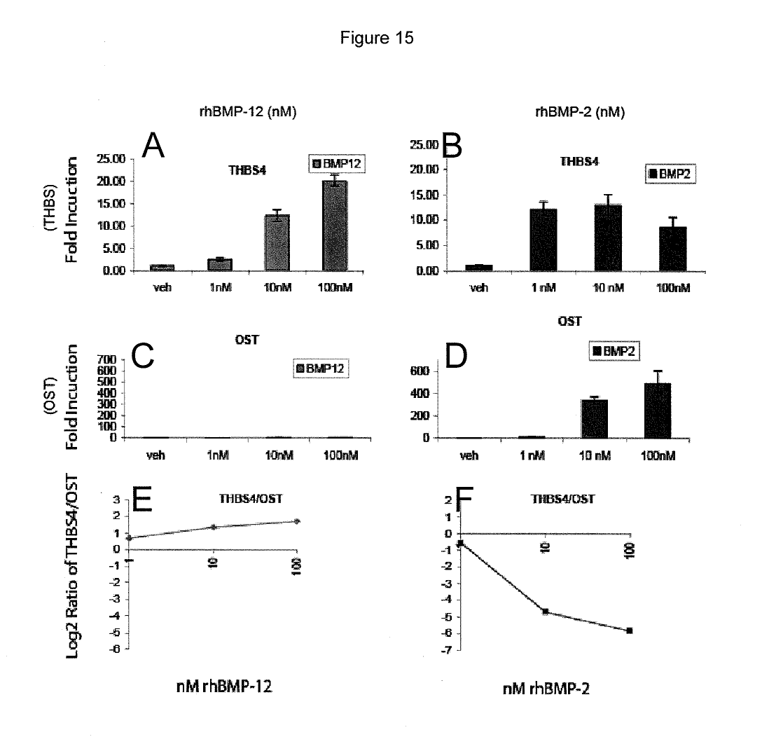

Cells at later passages have also shown similar gene expression trends as those observed at passage 3-5. FIG. 15 summarizes results for THBS4, OST, and THBS4/OST ratio for cells at passage 9 treated with rhBMP-12 and rhBMP-2. These cells showed a strong rhBMP-12 dose-dependent induction of THBS4 and little to no induction of OST (5 fold), while rhBMP-2 produced a strong induction of OST (500 fold). The THBS4/OST ratio shows a dose dependent increase to rhBMP-12, suggesting that cells up to at least passage 9 show reproducible activity.

### Example 7

### Freeze Thaw

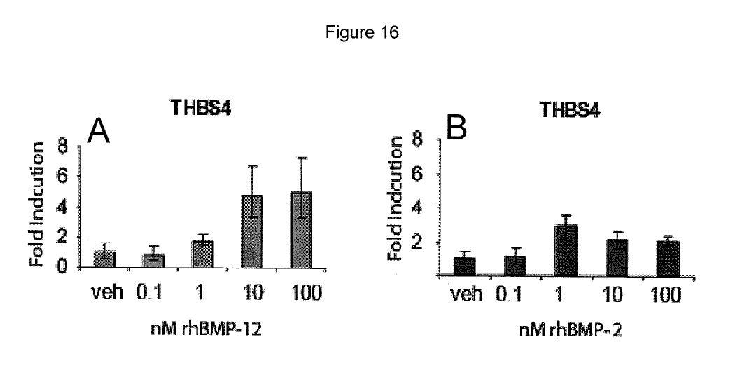

The majority of data presented was obtained using cells from the ATCC. The frozen stock was thawed once and continually passaged. We investigated whether a bank of cells can be frozen and then thawed for use at a later date. FIG. 16 shows data from cells that were frozen at passage 7, thawed at a later date, cultured to passage 10 and then assayed. Again, rhBMP-12 induced THBS4 mRNA in a dose-dependent manner, with relatively little osteocalcin induction (not shown), while rhBMP-2 produced a peak response in THBS levels and strong osteocalcin induction (not shown). Thus, cells can be refrozen and thawed at a later time for use in the methods of the invention.

### Example 8

### BMP-12, BMP-13, and MP-52 all Induce THBS4 Similarly

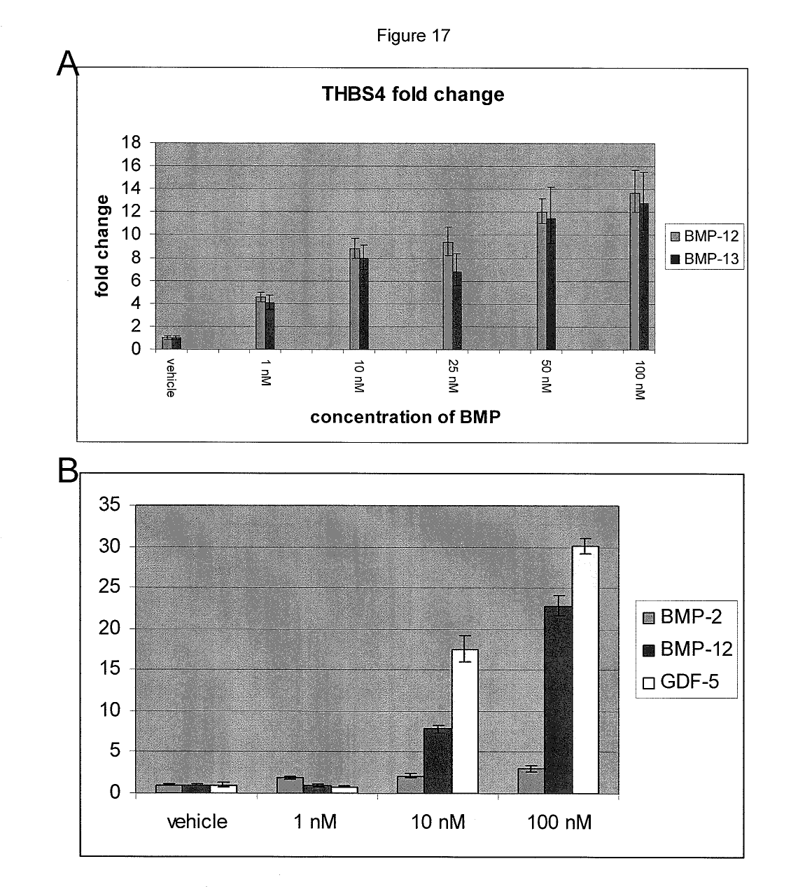

BMP-12, BMP-13, and MP-52 all similarly induced THBS4. FIG. 17a shows dose-response curves of C3H/10T1/2 cells, seeded at 2×10⁵ cells/well of a 12-well plate and incubated for 3 days at the indicated dose of BMP-12 or BMP-13. FIG. 17b shows that MP-52 (GDF5) elicited a response similar to BMP-12. These results demonstrate that all BMP-12-related proteins tested strongly induced THBS4 expression.

### Example 9

### Western Blot

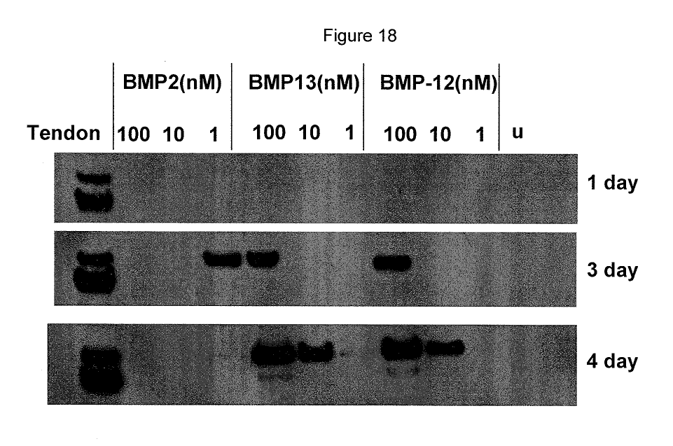

Supernatants of treated cells were concentrated using Microcon YM-10 spin columns (Millipore) and underwent SDS PAGE using NuPage MOPS 4-12% gels (Invitrogen). Gels were transferred to nitrocellulose membranes and THBS4 western blots were performed using goat anti-THBS4 polyclonal anti-bodies (R&D systems cat# AF2390) and anti-rabbit IgG HRP-conjugated secondary antibodies (Cell Signaling) Results are shown in FIG. 18. These experiments demonstrate that THBS4 induction in response to BMP-12-related proteins, but not osteogenic BMP-2, can be detected at both the protein or mRNA level.

### Example 10.1

### Branched DNA Assay

Cell culture conditions for Branched-DNA analysis were the same as those for RT-PCR analysis except that cells were harvested by aspirating off the cultured medium and then lysing in 800 μl diluted QuantiGene® Lysis Mixture with Proteinase K (DLM/PK) at 37° C. for 15 minutes (adapted from QuantiGene® 2.0 Reagent system User Manual. Panomics P/N13947 Rev. A 061106).

Branched DNA Hybridization analysis was carried out using custom design QuantiGene® 2.0 Reagent System probe sets from Panomics, Inc., which were used to hybridize to target mRNA sequence. These probe sets contain proprietary mixtures of three oligo construct types: Capture Extender oligos (CE), Label Extender (LE) oligos and Blocking Probes oligos (BL). The probe sets chosen for BMP-12 activity were as follows:

THBS4: Catalog Number SB-10037 (targeting bases 170-840 (exons 1-6) of THBS4 (accession number NM_{—}011582).
Osteocalcin (Bglap1): Catalog Number SB-11834 (targeting bases 24-460 of Bglap1 (accession number NM_{—}007541).
Beta-Glucuronidase (Gusb): Catalog Number SB-10015 (targeting bases 1514-1966) of Gusb (accession number NM_{—}010368).

### Example 10.2

### Branched-DNA Hybridization Analysis

Quantitation of target-specific RNA molecules in cell lysate was conducted using the QuantiGene® 2.0 Reagent System (adapted from QuantiGene® 2.0 Reagent system User Manual, Panomics part number 13947, Rev. A 061106). Working Probe Set mixes (CE, LE, BL) were prepared in Blocking Reagent and Diluted Lysis Mix (DLM) as follows: 1 μl of each oligo type, 10 μl Blocking reagent, and 187 ul DLM). 20 μl of a target Working Probe Set was added to capture plate wells followed by the addition of 40 μl DLM and 40 μl of sample cell lysate. The plate was sealed and target transcripts captured in a 16-20 hour incubation at 55° C. Washing and incubation steps were performed according to the manufacturer's protocol. Prior to assaying luminometric readout, the plate was allowed to return to room temperature in the dark. Data analysis was performed with Microsoft Excel. The blank signal was subtracted from the target signals. Sample cell lysates of each biological timepoint were evaluated in triplicate with the probeset for each gene. Results were then normalized to Gusb expression as a ratio of the sample THBS4 or OST signal by its Gusb signal. Normalized values were then averaged for each cohort, then expressed as a ratio of expression of each gene in each rhBMP treated sample compared to the expression of that gene in the control untreated sample. Results are shown in FIG. 19. These experiments demonstrate that THBS4 mRNA induction can be detected by a variety of methods, including qRT-PCR or branched DNA assays.

### Example 11

### Dynamic Range and Incubation Time

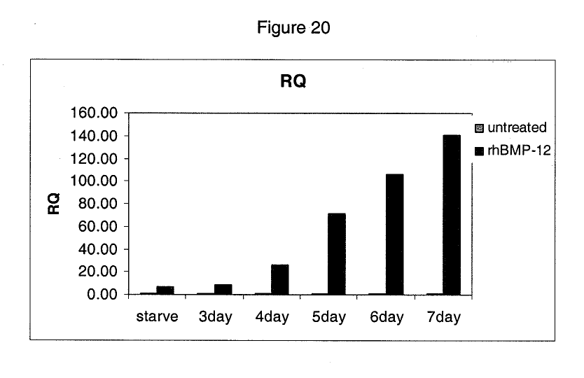

FIG. 20 shows the fold increase in THBS4 mRNA levels in C3H/10T1/2 cells treaded with 100 nM rh-BMP-12, versus control cells not incubated with BMP, for the indicated period of time. Cells were seeded at 2×10⁵ cells/well of a 12-well plate. The “starved” treatment group was incubated with serum-free medium for six hours before incubation with rhBMP-12 for three days. These experiments included bovine serum albumin (BSA) at a final concentration of 0.1% in the cell culture medium. These results demonstrate that the fold induction of THBS4 mRNA in cells treated with BMP-12, versus control cells not treated with BMP-12, continues to increase over time. Serum-starving cells before treatment did not greatly impact THBS4 induction after three days.

### Example 12

### ELISA

1. C3H/10T1/2 cells are plated at 2000 cells/cm2 in 12-well culture dishes. The cells are grown for four days in MEM with 10% FBS. The medium is changed to MEM with 1% FBS and either rhBMP-12 or rhBMP-13 at doses of 0, 10, 100, or 1000 nM. After 72 hours, culture supernatants are collected. The quantity of THBS4 protein in the supernatant is evaluated using a sandwich ELISA assay.

2. Titer plates are coated with anti-THBS4 capture antibody in phosphate-buffered saline (PBS) overnight at 4° C. After blocking for 1 hour with 2% BSA, the experimental samples are incubated in the titer plates at room temperature for two hours. After washing, an anti-THBS4 detecting antibody is added to the samples and incubated at room temperature for one hour. A horseradish peroxidase (HRP)-conjugated secondary antibody and tetramethylbenzidene (TMB) substrate are added to the sample for color detection. A standard curve ranging from 7.8-1000 ng/m¹ is constructed using recombinant human THBS4 (R&D Systems, Minneapolis, Minn. Cat# 2390-TH-050).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of detecting and/or measuring BMP-12-related protein activity in a test sample, comprising
i) contacting a BMP-12 responsive cell with the test sample; and
ii) measuring the expression level of at least one BMP-12-related-activity-marker, wherein a dose-responsive increase in the expression level of the BMP-12-related-activity-marker in the cell, compared to its expression level in a cell not contacted with the test sample, indicates the presence of BMP-12-related protein activity in the test sample.

2. The method of claim 1, wherein the at least one BMP-12-related-activity-marker is selected from the group consisting of thrombospondin-4 (THBS4), Tmtc4, AB112350, Birc5, Clu, Ccnb1, A1853363, Fut11, Gm2a, II17d, Matn2, Mia1, Mrps25, Mrps6, Ntrk2, Pbk, Pip, Prlr, Pcp4, Pcx, Rgs7, Ris2, 1500009L16Rik, Sfrp1, 2410017P07Rik, 2810417H13Rik, 3000004C01.Rik, 3110001A13Rik, Cytl1, 6230424C14Rik, 9130008F23Rik, 1133, Gas7, D430039N05Rik, SalI1, Sfrp1, Shcbp1, Trps1, and Scaper.

3. The method of claim 1, wherein the BMP-12-responsive cell is a mesenchymal cell.

4. The method of claim 3, wherein the BMP-12-responsive cell is capable of differentiating into a cell selected from a muscle cell, a bone cell, a fat cell, a ligament cell, a tendon cell, and a cartilage cell.

5. The method of claim 3, wherein the mesenchymal cell is selected from the group consisting of a C3H/10T1/2 cell, a 3T3L1 cell, a C2C12 cell, a murine Clone 14 cell, and a human primary mesenchymal stem cell.

6. The method of claim 1, wherein the test sample comprises at least one BMP-12-related protein.

7. The method of claim 6, wherein the test sample comprises at least one protein selected from the group consisting of BMP-12, BMP-13, and MP-52.

8. The method of claim 7, wherein the test sample comprises at least two proteins selected from the group consisting of BMP-12, BMP-13, and MP-52.

9. The method of claim 8, wherein the test sample comprises BMP-12, BMP-13, and MP-52.

10. The method of claim 6, wherein the test sample comprises a BMP-12 protein.

11. The method of claim 6, wherein the test sample comprises a BMP-13 protein.

12. The method of claim 6, wherein the test sample comprises a MP-52 protein.

13. The method of claim 1, wherein the BMP-12-related-activity-marker is THBS4.

14. The method of claim 13, wherein the THBS4 is a nucleic acid.

15. The method of claim 14, wherein the nucleic acid is an mRNA.

16. The method of claim 15, wherein the mRNA is detected by quantitative real-time polymerase chain reaction.

17. The method of claim 13, wherein the THBS4 is a peptide.

18. The method of claim 17, wherein the peptide is detected using an antibody.

19. The method of claim 18, wherein the antibody is R&D systems catalog number AF2390.

20. The method of claim 17, wherein the THBS4 is detected by an enzyme-linked immunosorbent assay (ELISA).

21. The method of claim 1, wherein the expression level of at least two BMP-12-related-activity-markers is measured.

22. The method of claim 21, wherein the expression level of at least three BMP-12-related-activity-markers is measured.

23. The method of claim 21 or claim 22, wherein one of the BMP-12-related-activity-markers is THBS4.

24. A method of detecting and/or measuring BMP-12-related protein activity in a test sample containing a BMP-12-related protein, comprising:
i) contacting a C3H/10T1/2 cell with the test sample; and
ii) detecting the expression level of thrombospondin-4 (THBS4) mRNA by quantitative real-time polymerase chain reaction,
wherein a dose-responsive increase in the expression level of THBS4 mRNA in the cell, compared to a cell not contacted with the test sample, indicates the presence of BMP-12-related protein activity in the test sample.

25. The method of claim 24, wherein the BMP-12-responsive cell is a mesenchymal cell.

26. The method of claim 25, wherein the BMP-12-responsive cell is capable of differentiating into a cell selected from a muscle cell, a bone cell, a fat cell, a ligament cell, a tendon cell, and a cartilage cell.

27. The method of claim 25, wherein the mesenchymal cell is selected from the group consisting of a C3H/10T1/2 cell, a 3T3L1 cell, a C2C12 cell, a murine Clone 14 cell, and a human primary mesenchymal stem cell.

28. The method of claim 24 wherein the test sample comprises BMP-12, BMP-13, or MP-52.

29. The method of claim 28, wherein the test sample comprises a BMP-12 protein.

30. The method of claim 28, wherein the test sample comprises a BMP-13 protein.

31. The method of claim 28, wherein the test sample comprises a MP-52 protein.

32. The method of claim 24 further comprising measuring the expression level of at least one BMP-12-related-activity-marker, in addition to THBS4.

33. The method of claim 32, further comprising measuring the expression level of at least two BMP-12-related-activity-markers, in addition to THBS4.

34. The method of claim 1, further comprising the step of measuring the expression level of at least one osteogenic-marker, wherein a dose-responsive increase in the ratio of the BMP-12-related-activity-marker expression level to the osteogenic-marker expression level in the cell, compared to the ratio in a cell not contacted with the test sample, indicates the presence of BMP-12-related protein activity in the test sample.

35. The method of claim 34, wherein the osteogenic-marker is osteocalcin (OST).

36. The method of claim 35, wherein the BMP-12-related-activity-marker is THBS4.

37. A method of detecting and/or measuring BMP-12-related protein activity in a test sample, comprising
i) contacting a BMP-12 responsive cell with the test sample;
ii) measuring the expression level of at least one BMP-12-related-activity-marker; and
iii) measuring the expression level of at least one osteogenic-marker,
wherein a dose-responsive increase in the ratio of the BMP-12-related-activity-marker expression level to the osteogenic-marker expression level in the cell, compared to the ratio in a cell not contacted with the test sample, indicates the presence of BMP-12-related protein activity in the test sample.

38. The method of claim 37, wherein the osteogenic-marker is osteocalcin (OST).

39. The method of claim 38, wherein the BMP-12-related-activity-marker is THBS4.


SEQUENCE LISTING (22 sequences)

SEQ ID NO: 1 - DNA, 23 bp, Artificial Sequence
  Description of Artificial Sequence Synthetic primer
tttgcgaaacaccattgctgaat

SEQ ID NO: 2 - DNA, 21 bp, Artificial Sequence
  Description of Artificial Sequence Synthetic primer
gcaccagtgtgtttggagttg

SEQ ID NO: 3 - DNA, 17 bp, Artificial Sequence
  Description of Artificial Sequence Synthetic primer
ccaagcttgtggtcctc

SEQ ID NO: 4 - DNA, 18 bp, Artificial Sequence
  Description of Artificial Sequence Synthetic primer
gcatgcgcagtgcattga

SEQ ID NO: 5 - DNA, 24 bp, Artificial Sequence
  Description of Artificial Sequence Synthetic primer
ccacatcctttccacagacatagc

SEQ ID NO: 6 - DNA, 17 bp, Artificial Sequence
  Description of Artificial Sequence Synthetic primer
caccacacacacacgtc

SEQ ID NO: 7 - DNA, 20 bp, Artificial Sequence
  Description of Artificial Sequence Synthetic primer
gactcctgtgacaccaacca

SEQ ID NO: 8 - DNA, 22 bp, Artificial Sequence
  Description of Artificial Sequence Synthetic primer
gctgggagctgttaatgactgt

SEQ ID NO: 9 - DNA, 17 bp, Artificial Sequence
  Description of Artificial Sequence Synthetic primer
catccccgtcactgtct

SEQ ID NO: 10 - DNA, 20 bp, Artificial Sequence
  Description of Artificial Sequence Synthetic primer
gctcagatcgaccccaactg

SEQ ID NO: 11 - DNA, 25 bp, Artificial Sequence
  Description of Artificial Sequence Synthetic primer
tcccttcaaagtcaactccgttaaa

SEQ ID NO: 12 - DNA, 16 bp, Artificial Sequence
  Description of Artificial Sequence Synthetic primer
ccgtgtacccaacagc

SEQ ID NO: 13 - DNA, 16 bp, Artificial Sequence
  Description of Artificial Sequence Synthetic primer
actcggtgcgcaacct

SEQ ID NO: 14 - DNA, 24 bp, Artificial Sequence
  Description of Artificial Sequence Synthetic primer
caattcaatggactctgggttctg

SEQ ID NO: 15 - DNA, 16 bp, Artificial Sequence
  Description of Artificial Sequence Synthetic primer
ccccagagccttttct

SEQ ID NO: 16 - DNA, 19 bp, Artificial Sequence
  Description of Artificial Sequence Synthetic primer
cggccctgagtctgacaaa

SEQ ID NO: 17 - DNA, 22 bp, Artificial Sequence
  Description of Artificial Sequence Synthetic primer
gccggagtctgttcactacctt

SEQ ID NO: 18 - DNA, 24 bp, Artificial Sequence
  Description of Artificial Sequence Synthetic probe
ccttcatgtccaagcaggagggca

SEQ ID NO: 19 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLNKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVPPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKAERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 20 - DNA, 3365 bp, Homo sapiens
ctggtccgtccaggctccttcccatcctcacacccgcgcctttctccctgcggccccggctcgctgctccagctgcccagctcttcccccgcccggccgcaccataaagcgcccggccgctgccgcggagcccagcagccagctccccagcaccgcacggcggggacgcgagcgcgcccccgacggcagcccggacgccgagcacgggtcacctgcggcgccggcccgggcgccgaccgaggttcaacgcacggcccggggacccccaggcggggccaacgccgccgtcgcccccggcctcgcggggagcaggaagagccaacatgctggccccgcgcggagccgccgtcctcctgctgcacctggtcctgcagcggtggctagcggcaggcgcccaggccaccccccaggtctttgaccttctcccatcttccagtcagaggctaaacccaggcgctctgctgccagtcctgacagaccccgccctgaatgatctctatgtgatttccaccttcaagctgcagactaaaagttcagccaccatcttcggtctttactcttcaactgacaacagtaaatattttgaatttactgtgatgggacgcttaaacaaagccatcctccgttacctgaagaacgatgggaaggtgcatttggtggttttcaacaacctgcagctggcagacggaaggcggcacaggatcctcctgaggctgagcaatttgcagcgaggggccggctccctagagctctacctggactgcatccaggtggattccgttcacaatctccccagggcctttgctggcccctcccagaaacctgagaccattgaattgaggactttccagaggaagccacaggacttcttggaagagctgaagctggtggtgagaggctcactgttccaggtggccagcctgcaagactgcttcctgcagcagagtgagccactggctgccacaggcacaggggactttaaccggcagttcttgggtcaaatgacacaattaaaccaactcctgggagaggtgaaggaccttctgagacagcaggttaaggaaacatcatttttgcgaaacaccatagctgaatgccaggcttgcggtcctctcaagtttcagtctccgaccccaagcacggtggtgcccccggctccccctgcaccgccaacacgcccacctcgtcggtgtgactccaacccatgtttccgaggtgtccaatgtaccgacagtagagatggcttccagtgtgggccctgccccgagggctacacaggaaacgggatcacctgtattgatgttgatgagtgcaaataccatccctgctacccgggcgtgcactgcataaatttgtctcctggcttcagatgtgacgcctgcccagtgggcttcacagggcccatggtgcagggtgttgggatcagttttgccaagtcaaacaagcaggtctgcactgacattgatgagtgtcgaaatggagcgtgcgttcccaactcgatctgcgttaatactttgggatcttaccgctgtgggccttgtaagccggggtatactggtgatcagataaggggatgcaaagcggaaagaaactgcagaaacccagagctgaacccttgcagtgtgaatgcccagtgcattgaagagaggcagggggatgtgacatgtgtgtgtggagtcggttgggctggagatggctatatctgtggaaaggatgtggacatcgacagttaccccgacgaagaactgccatgctctgccaggaactgtaaaaaggacaactgcaaatatgtgccaaattctggccaagaagatgcagacagagatggcattggcgacgcttgtgacgaggatgctgacggagatgggatcctgaatgagcaggataactgtgtcctgattcataatgtggaccaaaggaacagcgataaagatatctttggggatgcctgtgataactgcctgagtgtcttaaataacgaccagaaagacaccgatggggatggaagaggagatgcctgtgatgatgacatggatggagatggaataaaaaacattctggacaactgcccaaaatttcccaatcgtgaccaacgggacaaggatggtgatggtgtgggggatgcctgtgacagttgtcctgatgtcagcaaccctaaccagtctgatgtggataatgatctggttggggactcctgtgacaccaatcaggacagtgatggagatgggcaccaggacagcacagacaactgccccaccgtcattaacagtgcccagctggacaccgataaggatggaattggtgacgagtgtgatgatgatgatgacaatgatggtatcccagacctggtgccccctggaccagacaactgccggctggtccccaacccagcccaggaggatagcaacagcgacggagtgggagacatctgtgagtctgactttgaccaggaccaggtcatcgatcggatcgacgtctgcccagagaacgcagaggtcaccctgaccgacttcagggcttaccagaccgtggtcctggatcctgaaggggatgcccagatcgatcccaactgggtggtcctgaaccagggcatggagattgtacagaccatgaacagtgatcctggcctggcagtggggtacacagcttttaatggagttgacttcgaagggaccttccatgtgaatacccagacagatgatgactatgcaggctttatctttggctaccaagatagctccagcttctacgtggtcatgtggaagcagacggagcagacatattggcaagccaccccattccgagcagttgcagaacctggcattcagctcaaggctgtgaagtctaagacaggtccaggggagcatctccggaactccctgtggcacacgggggacaccagtgaccaggtcaggctgctgtggaaggactccaggaatgtgggctggaaggacaaggtgtcctaccgctggttcctacagcacaggccccaggtgggctacatcagggtacgattttatgaaggctctgagttggtggctgactctggcgtcaccatagacaccacaatgcgtggaggccgacttggcgttttctgcttctctcaagaaaacatcatctggtccaacctcaagtatcgctgcaatgacaccatccctgaggacttccaagagtttcaaacccagaatttcgaccgcttcgataattaaaccaaggaagcaatctgtaactgcttttcggaacactaaaaccatatatattttaacttcaattttctttagcttttaccaacccaaatatatcaaaacgttttatgtgaatgtggcaataaaggagaagagatcatttttaaaaaaaaaaaaaaa

SEQ ID NO: 21 - protein, 963 aa, Mus musculus
MPAPRAAAAAFLLLHLVLQPWQRTSAQATPQVFDLLPSSSQRLNPSALQPVLTDPTLHEVYLISTFKLQSKSSATIFGLYSSSDNSKYFEFTVMGRLNKAILRYLKNDGKIHLVVFNNLQLADGRRHRVLLRLSNLQRGDGSVELYLDCAQADSVRNLPRAFSGLTQNPESIELRTFQRKPQDFLEELKLVVRGSLFQVASLQDCFLQQSEPLAATSTGDFNRQFLGQMTQLNQLLGEVKDLLRQQVKETSFLRNTIAECQACGPLSFQSPTPNTLVPIAPPAPPTRPTRHCDSSPCFRGVRCTDTRDGFQCGPCPDGYTGNGITCSDVDECKYHPCYPGVRCVNLAPGFRCDACPVGFTGPMVQGVGINFAKTNKQVCTDVDECQNGACVLNSICINTLGSYRCGPCKPGYTGDQTRGCKTERSCRNPEQNPCSVHAQCIEERQGDVTCVCGVGWAGDGYVCGKDVDIDSYPDEELPCSARNCKKDNCKYVPNSGQEDADRDGIGDACDEDADGDGILNEQDNCVLTHNIDQRNSDKDIFGDACDNCRMVLNNDQKDTDGDGRGDACDDDMDGDGIKNILDNCPRVPNRDQQDRDGDDVGDACDSCPDVSNPNQSDVDNDLVGDSCDTNQDSDGDGHQDSTDNCPTVINSSQLDTDKDGIGDECDDDDDNDGIPDLVPPGPDNCRLVPNPAQEDSNNDGVGDICEADFDQDQVIDHIDVCPENAEITLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQSFDRLDN

SEQ ID NO: 22 - DNA, 3159 bp, Mus musculus
cccgaggccagcaaacagcgccctacgccggcaaagccacgccacattgtcgcctgcacagccctcagggagcagaagagtcaccatgccggccccacgcgcggccgcggccgccttcctcctgctgcacctggtcctgcagccgtggcagcggacgagcgcccaggccacaccccaggtctttgatcttctaccgtcctccagccagaggctgaaccccagcgccctgcagccagtcctgacagatcccaccctgcacgaagtctatctcatctccaccttcaagctgcagagtaaaagctcagccaccatctttggcctctactcctcaagtgacaacagcaaatactttgaattcactgtgatgggacggctgaacaaagccatcctccgctacctgaagaatgatggaaagatccatctggtggtgttcaacaacttgcagctggctgatgggaggcgccacagggttctcctgagactgagcaatttgcagagaggagatggctctgtagaactctacctggactgcgctcaggcggactcggtgcgcaacctccccagagccttttctggcctcacccagaacccagagtccattgaattgcggacattccagaggaagccccaggacttcttggaagagctgaagctggtggtgcggggctcgctgttccaggtggccagcctccaagactgcttcctgcagcagagtgagcctctggctgccacaagcacaggagactttaatcgacagttcttgggtcaaatgacacaactgaaccagctccttggagaggtgaaagatcttctgaggcagcaggtcaaggaaacatcgtttttgcgaaacaccattgctgaatgccaagcttgtggtcctctcagctttcagtccccaactccaaacacactggtgcccatagctcctccagcacccccaactcgcccaacacgccactgtgactccagcccttgtttccgaggtgtccgatgtacggacaccagggatggcttccagtgtggaccctgcccagatggttacacgggaaacgggatcacctgctctgacgtggatgagtgcaaataccatccctgctatccaggtgtgcgctgtgtgaatttggctccgggtttcagatgtgacgcctgtccagtaggtttcacagggcccatggtgcagggtgtcgggatcaactttgctaaaacaaacaagcaggtgtgcactgatgttgatgagtgtcagaatggagcgtgtgttctgaattctatctgcattaacactttgggatcttatcgctgtgggccttgcaaaccagggtacactggtgatcagacgaggggatgcaagactgaaaggagctgccggaacccagagcagaacccatgcagtgtgcatgcgcagtgcattgaggagcggcaaggggatgtgacgtgtgtgtgtggtgttggctgggctggtgacggctatgtctgtggaaaggatgtggacattgacagttaccctgatgaagaactgccatgctctgccaggaactgcaagaaggataactgcaagtatgtgccaaactctggccaggaagatgcagacagagatggcatcggagacgcgtgtgatgaggatgcagatggagatgggatcttgaatgaacaggacaactgtgtcctgactcacaatatagaccaaaggaacagtgataaagatatctttggggatgcctgtgacaactgccggatggtcctgaataatgaccagaaggatactgacggggatgggagaggagacgcctgtgatgacgacatggatggagatggaataaaaaacattttggacaactgtcccagagttcccaaccgtgaccagcaggatcgggatggtgatgatgtcggagacgcttgtgacagctgtcctgatgtcagtaaccctaaccagtcggatgtggataacgatctggttggggactcctgtgacaccaaccaagacagtgacggggatggccaccaggacagcacagacaactgccccacagtcattaacagctcccagctggacactgacaaagatgggatcggcgatgaatgtgatgacgatgacgataacgatggcatcccagacctggtgcctcctggcccagacaactgcaggctcgtccccaacccagcccaggaagatagcaacaatgacggggtaggagatatctgtgaagctgacttcgaccaggaccaggtcatcgatcacattgatgtgtgccctgagaatgcggagatcaccctgactgacttcagggcctaccagacagtggtcctggaccctgaaggggatgctcagatcgaccccaactgggtggtcctgaaccagggcatggaaattgtgcagaccatgaacagtgaccctggcctagctgttgggtacacggcatttaacggagttgactttgaagggaccttccacgtaaacacccagacagatgatgactatgctggctttatttttggttaccaagacagctccagcttctatgtagtcatgtggaagcaaacggagcagacatactggcaggctacaccgttccgtgcagtagcagaacctggtattcagctcaaggctgtgaagtctaaaactggcccaggggaacatctccgaaactccctgtggcacacaggggacaccagcgaccaggtcaggctgctgtggaaggactccaggaatgtgggctggaaggacaaggtctcctaccgctggttcctgcagcataggccccaggtgggctacatcagggtgcgcttttatgaaggctcggagttggtggctgactctggggtcaccatagacaccaccatgcgtggaggccgacttggtgtgttctgcttctctcaagaaaacatcatctggtccaacctcaagtaccgctgcaacgacaccataccagaggacttccaggagttccagacccagagttttgaccgcttggataattgagccaagaagcaatcccacaactgccttctcaaacaccaaagcaacatcttttttaaaacttcctataattctctttatattatcatggcttttcttttaccagcccaaatacgtcaaaactttttatatgaatgtggaaataaagaagagactatttctaataagaaaaaaaaaaaaaaaaa